SEQUENCE LISTING (24 sequences)

SEQ ID NO: 1 - protein, 235 aa, Homo sapiens
KVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 235 aa, Homo sapiens
KVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 3 - protein, 235 aa, Homo sapiens
KVDKRVESKYGPPCPPCPAPEFLGGPSVFLFPPKPKDTLMASRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 4 - protein, 235 aa, Homo sapiens
KVDKRVESKYGPPCPPCPAPEFLGGPSVFLFPPKPKDTLMNSRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 5 - protein, 235 aa, Homo sapiens
KVDKRVESKYGPPCPPCPAPEFLGGPSVFLFPPKPKDTLMASRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 6 - protein, 235 aa, Homo sapiens
KVDKRVESKYGPPCPPCPAPEFLGGPSVFLFPPKPKDTLMNSRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 7 - protein, 235 aa, Homo sapiens
KVDKRVESKYGPPCPPCPAPEFLGGPSVFLFPPKPKDTLMASRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 235 aa, Homo sapiens
KVDKRVESKYGPPCPPCPAPEFLGGPSVFLFPPKPKDTLMNSRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 235 aa, Homo sapiens
KVDKRVESKYGPPCPPCPAPEFLGGPSVFLFPPKPKDTLMASRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 235 aa, Homo sapiens
KVDKRVESKYGPPCPPCPAPEFLGGPSVFLFPPKPKDTLMNSRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 235 aa, Homo sapiens
KVDKRVESKYGPPCPPCPAPEFLGGPSVFLFPPKPKDTLMNSRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNAYTQKSLSLSLGK

SEQ ID NO: 12 - protein, 235 aa, Homo sapiens
KVDKRVESKYGPPCPPCPAPEFLGGPSVFLFPPKPKDTLMNSRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNEYTQKSLSLSLGK

SEQ ID NO: 13 - protein, 235 aa, Homo sapiens
KVDKRVESKYGPPCPPCPAPEFLGGPSVFLFPPKPKDTLMASRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNAYTQKSLSLSLGK

SEQ ID NO: 14 - protein, 235 aa, Homo sapiens
KVDKRVESKYGPPCPPCPAPEFLGGPSVFLFPPKPKDTLMASRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSPEEMTKNQVSLTCLVKGFYPSDIAVEWESNGQPENNYKTTPPVLDSDGSFFLYSRLTVDKSRWQEGNVFSCSVMHEALHNEYTQKSLSLSLGK

SEQ ID NO: 15 - protein, 778 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAKVDKRVESKYGPPCPPCPAPEFLGGPSVFLFPPKPKDTLMNSRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLSCAVKGFYPSDIAVEWESNGQPENNYKTTPPVLDSDGSFFLVSRLTVDKSRWQEGNVFSCSVMHEALHNQYTQKSLSLSLGK

SEQ ID NO: 16 - protein, 775 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASKVDKRVESKYGPPCPPCPAPEFLGGPSVFLFPPKPKDTLMNSRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLSCAVKGFYPSDIAVEWESNGQPENNYKTTPPVLDSDGSFFLVSRLTVDKSRWQEGNVFSCSVMHEALHNQYTQKSLSLSLGK

SEQ ID NO: 17 - protein, 781 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAKVDKKVEPKSCDKTHTCPPCPAPELLGGPSVFLFPPKPKDTLMNSRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNQYTQKSLSLSPGK

SEQ ID NO: 18 - protein, 778 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASKVDKKVEPKSCDKTHTCPPCPAPELLGGPSVFLFPPKPKDTLMNSRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNQYTQKSLSLSPGK

SEQ ID NO: 19 - protein, 873 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMNSRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNQYTQKSLSLSPGK

SEQ ID NO: 20 - protein, 870 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMNSRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNQYTQKSLSLSPGK

SEQ ID NO: 21 - protein, 596 aa, Artificial Sequence
  fusion protein
MKYLLPTAAAGLLLLAAQPAMADDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMNSRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSPEEMTKNQVSLWCLVKGFYPSDIAVEWESNGQPENNYKTTPPVLDSDGSFFLYSRLTVDKSRWQEGNVFSCSVMHEALHNQYTQKSLSLSLGK

SEQ ID NO: 22 - protein, 598 aa, Artificial Sequence
  fusion protein
MKYLLPTAAAGLLLLAAQPAMADIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMNSRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNQYTQKSLSLSPGK

SEQ ID NO: 23 - protein, 467 aa, Artificial Sequence
  fusion protein
MEFGLSWVFLVALLRGVQCEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMNSRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNQYTQKSLSLSPGK

SEQ ID NO: 24 - protein, 236 aa, Artificial Sequence
  fusion protein
MKYLLPTAAAGLLLLAAQPAMADIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2022/0195051 A1
(43) Pub. Date: Jun. 23, 2022

(54) Fc-MODIFIED BIOLOGICALS FOR LOCAL DELIVERY TO COMPARTMENT, IN PARTICULAR TO THE CNS

(71) Applicant: Universität Zürich, Zürich (CH)

(72) Inventors: Johannes vom BERG, Zürich (CH); Damien MORGER, Zürich (CH); Linda SCHELLHAMMER, Schlieren (CH); Michal BEFFINGER, Schlieren (CH); Thorsten BUCH, Schlieren (CH)

(21) Appl. No.: 17/599,921

(22) PCT Filed: Mar. 27, 2020
(86) PCT No.: PCT/EP2020/058876

§ 371 Date: Sep. 29, 2021

(30) Foreign Application Priority Data
Mar. 29, 2019 (EP) 19166231.1
Jul. 16, 2019 (EP) 19186619.3

(51) Int. Cl. C07K 16/28; A61P 35/00; C07K 14/54

(52) U.S. Cl. CPC C07K 16/2827; C07K 16/2866; A61K 2039/505; C07K 14/5434; C07K 2317/92; A61P 35/00

(57) ABSTRACT

A polypeptide comprising a crystallizable fragment (Fc) region of IgG for use in prevention or treatment of a disease, particularly a disease affecting the central nervous system. The polypeptide is administered locally to the affected compartment, in particular to the central nervous system. The Fc region bears a modification resulting in reduced affinity to the neonatal Fc receptor (FcRn), resulting in an increased brain to serum concentration of the polypeptide.

The present invention relates to locally delivered biological pharmaceuticals characterized by an Fc polypeptide having a lowered affinity towards the neonatal Fc receptor (FcRn), in particular for use in neurological diseases.

Currently the incidence of neurological diseases in Europe and USA is over 200 cases per 100′000 citizens and due to an ageing population it is expected to increase further. Advances in pre-clinical research offer many promising targets for local treatment of neurological diseases, including cytokine therapies (e.g. IL-12 for brain tumors, IL-10 for MS), as well as neutralizing antibodies (e.g. against interleukin (IL)-12/23p40 in MS, or against tumor necrosis factor α (TNFα) in Parkinson's Disease and Alzheimer's Disease) or immune checkpoint blocking molecules (e.g. blocking PD-1/PD-L1 axis in brain tumors).

Immunotherapy is one of the most promising directions in brain tumor treatment. Interleukin (IL)-12 is a pro-inflammatory cytokine and has a powerful anti-tumor effect on brain tumors in preclinical models. Based on promising preclinical results, clinical testing was rapidly initiated in the late 90s as an intravenously (i.v.) applied systemic treatment using IL-12. However, a phase II clinical trial reported severe adverse events, with 12 out of 17 patients hospitalized and two patients dead. These adverse effects have since been attributed to the rapid induction of high systemic levels of interferon (IFN)-γ, an IL-12 downstream effector cytokine.

Given the toxicity of systemically applied IL-12 and the need for a high concentration at the tumor site, a tight control over IL-12 levels in the tissue is a mandatory prerequisite of clinical applications. Local administration to the brain has recently become possible by using novel neurosurgical techniques, such as convection enhanced delivery (CED). Local intracranial delivery does however not preclude subsequent systemic leakage.

Murine IL-12Fc, a single chain fusion protein of IL-12 and the crystallisable fragment (Fc) of immunoglobulin G, shows increased pharmacostability, bioavailability and a reduced passive leakage from the brain compared to unmodified recombinant IL-12. Following local delivery to the brain, it is however actively exported across the blood brain barrier (BBB) by the neonatal Fc receptor (FcRn), a receptor that mediates export of all proteins comprising an Fc region from the cerebrospinal fluid. FcRn is also active in endothelial cells and in red pulp macrophages, where it prevents degradation and prolongs serum half-life live of Fc containing molecules and serum albumin. Compared to unmodified IL-12, IL-12Fc thus shows an increased systemic accumulation.

The IgG Fc residues known to be involved in FcRn binding (isoleucine 253—I253, histidine 310—H310 and histidine 435—H435) as well as the pH dependence of the interaction between these residues and FcRn are known from the state of the art (Pyzik et al. Frontiers in Immunology (2019) 10:1540).

For example, Bitonti et al. reported that mutating the residues I253, H310 and H435 in the Fc domain of wild-type IgG to alanine 253—A253, A310 and A435 (AAA), respectively, leads to abrogation of FcRn binding at pH 6 (Bitonti et al. Proceedings of the National Academy of Sciences (2004) 101(26):9763-9768).

However, the substitution of an amino acid to alanine is a common biochemical method of screening for functional roles at given positions within a protein of interest. Apart from this one particular mutation (AAA), the article does not disclose any other mutations from which conclusions could be drawn about the resulting binding properties to FcRn. Moreover, the article deals with FcRn-mediated transport of a Fc fusion protein comprising erythropoietin (Epo), a glycoprotein hormone drug that stimulates red blood cell production, in the lung of non-human primates. The article remains silent with regard to the applicability of the results to a fusion polypeptide comprising IL-12 and the administration of Fc fusion polypeptides to the brain, respectively.

There are publications that actually deal with fusion polypeptides comprising IL-12 and ways to increase their serum half-life.

For example, Jung et al. describe the generation and anti-tumor activity of a fusion polypeptide comprising IL-12 and human IgG4-based heterodimeric Fc bearing an A107 mutation pair which affords reduced affinity to Fcγ receptors (Jung et al., Oncoimmunology, 7(7):e1438800).

However, as the Fc gamma receptor (FcγR) family is a functional grouping of proteins characterized by binding to the constant region of antibodies, i.e. the Fc part, albeit with differences in structure, non-overlapping binding sites at the Fc part, localization in different compartments of the cell (intracellular vs. extracellular), pH dependent binding (acidic vs. neutral) and overall function, it is apparent that FcRn cannot be equated with FcγRs.

In another example from the state of the art, a comparison is made between recombinant IL-12 and IL-12Fc with regard to tissue retention and leakage into the systemic circulation (Beffinger et al., Neuro-Oncology (2017), 19(suppl-.6), vi273). Therein, the authors state that IL-12Fc showed a higher brain concentration 24 hours after intracranial application compared to recombinant IL-12.

However, the study does not disclose a fusion polypeptide bearing a mutation in the Fc region of IgG or an effect on the binding to FcRn.

Cooper et al. studied the role of FcRn in IgG efflux from rat brains upon local delivery of two variants of a recombinant human IgG1 mAb that either had increased FcRn binding (IgG1 asparagine 434 to alanine, N434A) or decreased FcRn binding (IgG1 histidine 435 to alanine, H435A) compared to wild-type Fc of IgG1 (Cooper et al. Brain Research (2013) 1534:13-21). The mutants were obtained by incorporating mutations at the 434 and 435 amino acid positions, respectively. The study has been conducted in rats, using human antibodies.

With regard to binding properties of Fc mutants towards the mouse and human forms of FcRn, Andersen et al. disclosed five distinct Fc mutants with mutations at the level of Ile253, His310 and His435, i.e. H435Q, H435R, H310A, I253A, and H310A/H435Q (Andersen et al. Journal of Biological Chemistry (2012) 287(27):22927-22937). The variant featuring the lowest affinity for human FcRn was the mutant bearing both H310A and H435Q mutations (IAQ).

Even though the last two studies mentioned herein demonstrated that FcRn plays an important role in the effux of IgGs from rat brains and disclosed distinct mutants with reduced affinity to FcRn, respectively, neither of these studies serves as a basis for assessing how the presence of IL-12Fc would have affected binding to FcRn. Moreover, the concept of generating a maximal brain-to-blood concentration gradient is not disclosed.

Based on the above mentioned state of the art, the objective of the present invention is to provide means and methods to extend the therapeutic window of pharmaceuticals that are locally delivered to a specific compartment, in particular the brain, and preventing both export from the said compartment, in particular the brain, and systemic accumulation, thereby increasing the compartment-to-serum ratio, in particular the brain-to-serum ratio. This objective is attained by the claims of the present specification.

In the context of the present specification, the term crystallizable fragment (Fc) region refers to a fraction of an IgG antibody comprising two identical heavy chain fragments covalently linked by disulfide bonds or to a single heavy chain fragment. The heavy chain fragments are comprised of constant domains (a C_{H}2 and a C_{H}3 domain in IgG antibody isotypes).

In the context of the present specification, the EU numbering system (Edelman et al. Proceedings of the National Academy of Sciences of the United States of America (1969) 63(1):78-85) is used for the numbering of amino acid residues in the Fc region. The EU numbering scheme is a widely adopted standard for numbering the residues in an antibody in a consistent manner.

Amino acid sequences are given from amino to carboxyl terminus. Capital letters for sequence positions refer to L-amino acids in the one-letter code (Stryer, Biochemistry, 3^{rd} ed. p. 21). Lower case letters for amino acid sequence positions refer to the corresponding D- or (2R)-amino acids.

Amino acid residues I253, H310 and H435 are located at the C_{H}2-C_{H}3 domain interface and are—with the exception of R435 in human IgG3—conserved across IgG subclasses within species and between IgG molecules found in both rodents and humans (Miyakawa et al. RNA (2008) 14:1154-1163). According to the present invention, the modified Fc regions or fragments thereof may be derived from IgG1, IgG2 or IgG4 immunoglobulins and should include at least amino acid residues 253, 310 and 435 of the Fc domain of immunoglobulin G (IgG) according to the EU numbering system.

In the context of the present specification, IL-12 refers to interleukin 12.

In the context of the present specification, hIL-12 relates to human IL-12.

In the context of the present specification, mIL-12 relates to murine IL-12.

In the context of the present specification, rhIL-12 relates to recombinant human IL-12.

In the context of the present specification, rmIL-12 relates to recombinant murine IL-12.

In the context of the present specification, IL-12Fc WT relates to IL-12 linked to a wild type, non-modified Fc region, in particular by fusion of p40 with p35 by means of a Gly-Ser-linker or by addition of an IgG4 tag.

In the context of the present specification, mIL-12hFc WT relates to murine IL-12 linked to a human wild type Fc region of IgG4 containing S228P mutation.

In the context of the present specification, mIL-12hFc NHQ relates to murine IL-12 linked to a human wild type Fc region of IgG4 containing serine 228 to proline—S228P, as well as NHQ mutations.

In the context of the present specification, mIL-12hFc:anti-PD-L1 bifunctional molecule relates to murine IL-12 linked to a human IgG1 Fc and dimerized with a half-molecule (one heavy and one light chain) of a fully human PD-L1 binding IgG1 antibody. The Fc part of the resulting molecule contains the NHQ mutations.

In the context of the present specification, FcRn^{tg} relates to a mouse strain lacking functional murine FcRn and carrying a transgene for expression of the human FcRn α-chain under the control of natural human regulatory elements described by the allele symbol Tg(FCGRT)32Dcr.

In the context of the present invention, an IL-12 polypeptide is a polypeptide having an amino acid sequence comprising the sequence of p35 (Uniprot ID 29459) or a functional homologue thereof and comprising the sequence of p40 (Uniprot ID29460) or a functional homologue thereof. In one embodiment, the IL-12 polypeptide has an amino acid sequence comprising both p35 and p40 sequences or homologues thereof as part of the same continuous amino acid chain. In said continuous amino acid chain only the N-terminal polypeptide (p40) functional homologue retains the signal peptide. In another embodiment, the IL-12 polypeptide comprises two distinct amino acid chains, one comprising the p35 sequence and another one comprising the p40 sequence, both having individual signal peptides. The IL-12 polypeptide has a biological activity of IL-12. A biological activity of IL-12 in the context of the present invention comprises the stimulation of NK or T cells by said IL-12 polypeptide, most prominently the stimulation of T effector cells acting through perforin.

In the context of the present specification, the terms sequence identity and percentage of sequence identity refer to the values determined by comparing two aligned sequences. Methods for alignment of sequences for comparison are well-known in the art. Alignment of sequences for comparison may be conducted by the local homology algorithm of Smith and Waterman, Adv. Appl. Math. 2:482 (1981), by the global alignment algorithm of Needleman and Wunsch, J. Mol. Biol. 48:443 (1970), by the search for similarity method of Pearson and Lipman, Proc. Nat. Acad. Sci. 85:2444 (1988) or by computerized implementations of these algorithms, including, but not limited to: CLUSTAL, GAP, BESTFIT, BLAST, FASTA and TFASTA. Software for performing BLAST analyses is publicly available, e.g., through the National Center for Biotechnology-Information (http://blast.ncbi.nlm.nih.gov/).

One example for comparison of amino acid sequences is the BLASTP algorithm that uses the default settings: Expect threshold: 10; Word size: 3; Max matches in a query range: 0; Matrix: BLOSUM62; Gap Costs: Existence 11, Extension 1; Compositional adjustments: Conditional compositional score matrix adjustment. One such example for comparison of nucleic acid sequences is the BLASTN algorithm that uses the default settings: Expect threshold: 10; Word size: 28; Max matches in a query range: 0; Match/Mismatch Scores: 1.-2; Gap costs: Linear. Unless otherwise stated, sequence identity values provided herein refer to the value obtained using the BLAST suite of programs (Altschul et al., J. Mol. Biol. 215:403-410 (1990)) using the above identified default parameters for protein and nucleic acid comparison, respectively.

In the context of the present specification, IL-10 refers to interleukin 10. In certain embodiments, IL-10 is employed in the treatment of inflammation, autoimmune inflammation, dementia or stroke. In certain embodiments, neutralizing IL-10 is employed in the treatment of pulmonary paracoccidioidomycosis.

In the context of the present specification, IL-2 refers to interleukin 2. In certain embodiments, IL-2 is employed in the treatment of cancer and infectious diseases.

In the context of the present specification, IL-7 refers to interleukin 7. In certain embodiments, IL-7 is employed in the treatment of cancer and infectious diseases.

In the context of the present specification, IFNγ refers to interferon gamma. In certain embodiments, IFNγ is employed in the treatment of cancer and infectious diseases.

In the context of the present specification, IL-15 refers to interleukin 15. In certain embodiments, IL-15 is employed in the treatment of cancer and infectious diseases.

In the context of the present specification, IL-23 refers to interleukin 23. In certain embodiments, IL-23 is employed in the treatment of cancer and infectious diseases.

In the context of the present specification, TNFα refers to tumor necrosis factor alpha, also known as cachexin, or cachectin. In certain embodiments, TNFα is employed in the treatment of cancer and infectious diseases. In certain embodiments, blocking TNFα is employed in the treatment of inflammation, autoimmune inflammation and arthritis. In certain embodiments, blocking of TNFα is employed in the treatment of uveitis. In certain embodiments, blocking of TNFα is employed in the treatment of rheumatoid arthritis. In certain embodiments, blocking of TNFα is employed in the treatment of sarcoidosis. In certain embodiments, blocking TNFα is employed in the treatment of cystic fibrosis.

In the context of the present specification, CTLA-4 refers to cytotoxic T-lymphocyte-associated protein 4, also known as CD152. In certain embodiments, blocking CTLA-4 is employed in the treatment of cancer. In certain embodiments, blocking of CTLA-4 is employed in the treatment of lung cancer.

In the context of the present specification, TGFβ refers to transforming growth factor beta. In certain embodiments, blocking TGFβ is employed in the treatment of cancer and infectious diseases. In certain embodiments, TGFβ is employed in the treatment of inflammation, autoimmune inflammation, dementia and stroke. In certain embodiments, TGFβ antagonist is employed in the treatment of cystic fibrosis.

In the context of the present specification, TGFα refers to transforming growth factor alpha. In certain embodiments, a TGFα antagonist is employed in the treatment of cystic fibrosis.

In the context of the present specification, TGFβRII refers to transforming growth factor beta receptor II. In certain embodiments, blocking TGFβRII or using TGFβRII-Fc is employed in the treatment of cancer and infectious diseases.

In the context of the present specification, GDNF refers to glial cell line-derived neurotrophic factor. In certain embodiments, GDNF is employed in the treatment of multiple sclerosis, Parkinson's disease, dementia, stroke and hereditary disorders.

In the context of the present specification, IL-35 refers to interleukin 35. In certain embodiments, IL-35 is employed in the treatment of inflammation, autoimmune inflammation, dementia and stroke.

In the context of the present specification, CD95 refers to Fas, also known as FasR, apoptosis antigen 1, APO-1, APT, or TNFR superfamily member 6. In certain embodiments, blocking CD95 is employed in the treatment of cancer.

In the context of the present specification, IL-1RA refers to Interleukin 1 receptor antagonist. In certain embodiments, IL-1RA is employed in the treatment of inflammation, autoimmune inflammation, rheumatoid arthritis, gout, pseudogout dementia and stroke. In certain embodiments, blocking of IL-1RA is employed in the treatment of rheumatoid arthritis.

In the context of the present specification, IL-4 refers to interleukin 4. In certain embodiments, IL-4 is employed in the treatment of inflammation, autoimmune inflammation, dementia and stroke.

In the context of the present specification, IL-13 refers to interleukin 13. In certain embodiments, IL-13 is employed in the treatment of inflammation, autoimmune inflammation, dementia and stroke. In certain embodiments, neutralizing anti-IL-13 is employed in the treatment of severe uncontrolled asthma. In certain embodiments, blocking and/or neutralizing IL-13 is employed in the treatment of chronic rhinosinusitis with nasal polyps. In certain embodiments, an IL-13 antagonist is employed in the treatment of idiopathic pulmonary fibrosis.

In the context of the present specification, TSLP refers to thymic stromal lymphopoietin, a protein belonging to the cytokine family. In certain embodiments, neutralizing TSLP is employed in the treatment of allergic asthma. In certain embodiments, blocking and/or neutralizing TSLP is employed in the treatment of chronic rhinosinusitis with nasal polyps.

In the context of the present specification, SIRPα refers to signal regulatory protein alpha. In certain embodiments, SIRPα is employed in the treatment of cancer.

In the context of the present specification, G-CSF refers to granulocyte-colony stimulating factor (G-CSF or GCSF), also known as colony-stimulating factor 3 (CSF 3). In certain embodiments, G-CSF is employed in the treatment of cancer.

In the context of the present specification, GM-CSF refers to granulocyte-macrophage colony-stimulating factor (GM-CSF), also known as colony-stimulating factor 2 (CSF2). In certain embodiments, GM-CSF is employed in the treatment of cancer. In certain embodiments, blocking GM-CSF is employed in the treatment of multiple sclerosis.

In the context of the present specification, GM-CSFR refers to granulocyte-macrophage colony-stimulating factor receptor (GM-CSFR), also known as CD116 (Cluster of Differentiation 116), a receptor for granulocyte-macrophage colony-stimulation, which stimulates the production of white blood cells. In certain embodiments, blocking GM-CSFR is employed in the treatment of rheumatoid arthritis.

In the context of the present specification, OX40L refers to ligand for OX40, also known as ligand for CD134. In certain embodiments, OX40L is employed in the treatment of cancer.

In the context of the present specification, CD80 refers to B7-1, also known as B7.1. In certain embodiments, CD80 is employed in the treatment of cancer.

In the context of the present specification, CD86 refers to B7-2, also known as B7.2. In certain embodiments, CD86 is employed in the treatment of cancer.

In the context of the present specification, GITRL refers to TNFSF18, AITRL, TL6, TNLG2A, TNF superfamily member 18. In certain embodiments, GITRL is employed in the treatment of cancer.

In the context of the present specification, 4-1BBL refers to ligand for 4-1BB, also known as ligand for ILA or ligand for CD137 or ligand for TNFR superfamily member 9. In certain embodiments, 4-1BB is employed in the treatment of cancer.

In the context of the present specification, EphrinA1 refers to EFNA1. In certain embodiments, EphrinA1 is employed in the treatment of cancer.

In the context of the present specification, EphrinB2 refers to EFNB2. In certain embodiments, EphrinB2 is employed in the treatment of cancer.

In the context of the present specification, EphrinB5 refers to EFNB5. In certain embodiments, EphrinB5 is employed in the treatment of cancer.

In the context of the present specification, PD-L1 refers to programmed death-ligand 1, also known as CD274 or B7 homolog 1 or B7-H1. In certain embodiments, PD-L1 blockade is employed in the treatment of cancer. In certain embodiments, blocking of PD-L1 is employed in the treatment of uveal melanoma. In certain embodiments, blocking of PD-1 is employed in the treatment of lung cancer.

In the context of the present specification, histone refers to proteins belonging to the histone families H1/H5, H2A, H2B, H3, and H4. In certain embodiments, binding histone is employed in the treatment of cancer.

In the context of the present specification, CXCL10 refers to C-X-C motif chemokine 10, also known as Interferon gamma-induced protein 10 (IP-10) or small-inducible cytokine B10. In certain embodiments, CXCL10 is employed in the treatment of cancer.

In the context of the present specification, PD-1 refers to programmed cell death protein 1, also known as CD279. In certain embodiments, binding PD-1 is employed in the treatment of cancer.

In certain other embodiments, binding PD-1 is employed in the treatment of dementia. In certain embodiments, blocking of PD-1 is employed in the treatment of uveal melanoma. In certain embodiments, blocking of PD-1 is employed in the treatment of lung cancer.

In the context of the present specification, TREM2 refers to triggering receptor expressed on myeloid cells 2. In certain embodiments, blocking TREM2 is employed in the treatment of inflammation, autoimmune inflammation, dementia and stroke.

In the context of the present specification, IL-6 refers to interleukin 6. In certain embodiments, blocking IL-6 is employed in the treatment of inflammation, autoimmune inflammation, dementia and stroke.

In the context of the present specification, IL-6R refers to interleukin 6 receptor. In certain embodiments, blocking IL-6R is employed in the treatment of inflammation, autoimmune inflammation, rheumatoid arthritis, juvenile idiopathic arthritis and adult-onset Still's disease. In certain embodiments, blocking and/or neutralising IL-6R is employed in the treatment of corona virus disease 2019 (COVID-19) and/or diseases caused by severe acute respiratory syndrome coronavirus (SARS-CoV).

In the context of the present specification, Cx3cr1 refers to CX3C chemokine receptor 1, also known as the fractalkine receptor or G-protein coupled receptor 13 (GPR13). In certain embodiments, binding Cx3cr1 is employed in the treatment of cancer, dementia, inflammation, autoimmune inflammation and stroke.

In certain embodiments, blocking CD27 is employed in the treatment of inflammation or autoimmune inflammation.

In certain embodiments, activating CD27 is employed in the treatment of cancer.

In certain embodiments, blocking CD25 is employed in the treatment of inflammation, autoimmune inflammation and multiple sclerosis.

In certain embodiments, binding CD25 is employed in the treatment of cancer.

In certain embodiments, activating CD28 is employed in the treatment of cancer.

In the context of the present specification, Nogo-A refers to neurite outgrowth inhibitor, also known as NOGO or NSP or NSP-CL Reticulon 4. In certain embodiments, blocking Nogo-A is employed in the treatment of autoimmune inflammation, traumatic CNS injury and stroke.

In the context of the present specification, IL-12Rb1 refers to interleukin-12 receptor beta 1 subunit. In certain embodiments, blocking IL-12Rb1 is employed in the treatment of inflammation, autoimmune inflammation, dementia and stroke.

In the context of the present specification, CD47 refers to integrin associated protein (IAP). In certain embodiments, blocking CD47 is employed in the treatment of cancer.

In the context of the present specification, CD147 refers to basigin (BSG), also known as extracellular matrix metalloproteinase inducer (EMMPRIN). In certain embodiments, blocking CD147 is employed in the treatment of corona virus disease 2019 (COVID-19). In certain embodiments, blocking CD147 is employed in the treatment of diseases caused by severe acute respiratory syndrome coronavirus (SARS-CoV). In the context of the present specification, EGFR refers to epidermal growth factor receptor, also known as ErbB-1. In certain embodiments, blocking EGFR is employed in the treatment of cancer.

In the context of the present specification, EGFRvIII refers to vIII mutant of epidermal growth factor receptor, also known as vIII mutant of ErbB-1. In certain embodiments, blocking EGFRvIII is employed in the treatment of cancer.

In the context of the present specification, Her2 refers to receptor tyrosine-protein kinase erbB-2, also known as CD340 or proto-oncogene Neu. In certain embodiments, blocking Her2 is employed in the treatment of cancer.

In the context of the present specification, PDGFR refers to platelet-derived growth factor receptors (PDGF-R). In certain embodiments, blocking PDGF-R is employed in the treatment of cancer.

In the context of the present specification, FGFR refers to fibroblast growth factor receptor. In certain embodiments, blocking FGFR is employed in the treatment of cancer.

In the context of the present specification, IL-4RA refers to interleukin 4 receptor, also known as IL-4R or CD124. In certain embodiments, blocking IL-4RA is employed in the treatment of cancer. In certain embodiments, blocking IL-4R is employed in the treatment of asthma.

In the context of the present specification, TfR refers to transferrin receptor. In certain embodiments, binding TfR is employed in the treatment of inflammation, autoimmune inflammation, dementia, traumatic CNS injury, cancer and stroke.

In the context of the present specification, LfR refers to lactoferrin receptor, also known as omentin or intestinal lactoferrin receptor. In certain embodiments, binding LfR is employed in the treatment of inflammation, autoimmune inflammation, dementia, traumatic CNS injury, cancer and stroke.

In the context of the present specification, IR refers to insulin receptor. In certain embodiments, binding IR is employed in the treatment of inflammation, autoimmune inflammation, dementia, traumatic CNS injury, cancer and stroke.

In the context of the present specification, LDL-R refers to low-density lipoprotein receptor. In certain embodiments, binding LDL-R is employed in the treatment of inflammation, autoimmune inflammation, dementia, traumatic CNS injury, cancer and stroke.

In the context of the present specification, LRP-1 refers to low density lipoprotein receptor-related protein 1 (LRP1), also known as alpha-2-macroglobulin receptor (A2MR) or apolipoprotein E receptor (APOER) or CD91. In certain embodiments, binding LRP-1 is employed in the treatment of inflammation, autoimmune inflammation, dementia, traumatic CNS injury, cancer and stroke.

In the context of the present specification, CD133 refers to prominin-1. In certain embodiments, binding CD133 is employed in the treatment of cancer.

In the context of the present specification, CD111 refers to poliovirus receptor-related 1 (PVRL1), also known as nectin-1. In certain embodiments, binding CD111 is employed in the treatment of cancer.

In the context of the present specification, VEGFR refers to receptors for vascular endothelial growth factor. In certain embodiments, blocking VEGFR is employed in the treatment of cancer or wet AMD, diabetic macular edema or retinitis pigmentosa.

In the context of the present specification, VEGF-A refers to vascular endothelial growth factor A. In certain embodiments, blocking VEGF-A is employed in the treatment of cancer or wet AMD, diabetic macular edema, retinitis pigmentosa or chronic haemophilic synovitis.

In the context of the present specification, Ang-2 refers to angiopoietin 2. In certain embodiments, blocking VEGF-A is employed in the treatment of cancer or wet AMD, diabetic macular edema or retinitis pigmentosa.

In the context of the present specification, IL-10R refers to interleukin 10 receptor, also known as receptor for cytokine synthesis inhibitory factor. In certain embodiments, blocking IL-10R is employed in the treatment of cancer.

In the context of the present specification, IL-13Rα2 refers to interleukin-13 receptor subunit alpha-2, also known as CD213A2. In certain embodiments, binding IL-13Rα2 is employed in the treatment of cancer. In certain embodiments, IL-13Rα2 is employed in the treatment of cancer. In certain embodiments, binding α-synuclein is employed in the treatment of Parkinson's disease.

In the context of the present specification, CSF1R refers to colony stimulating factor 1 receptor (CSF1R), also known as macrophage colony-stimulating factor receptor (M-CSFR), and CD115. In certain embodiments, blocking CSF1R is employed in the treatment of cancer.

In the context of the present specification, GITR refers to glucocorticoid-induced TNFR-related protein, also known as TNFR superfamily member 18 (TNFRSF18) or activation-inducible TNFR family receptor or AITR. In certain embodiments, binding GITR is employed in the treatment of cancer.

In the context of the present specification, CD22 refers to cluster of differentiation-22. In certain embodiments, blocking CD22 is employed in the treatment of neurodegenerative disease, autoimmune inflammation, dementia and stroke.

In the context of the present specification, TIM-3 refers to T-cell immunoglobulin and mucin-domain containing-3, also known as hepatitis A virus cellular receptor 2 (HAVCR2). In certain embodiments, blocking TIM-3 is employed in the treatment of cancer.

In the context of the present specification, LAG-3 refers to lymphocyte-activation gene 3. In certain embodiments, blocking LAG-3 is employed in the treatment of cancer. In certain embodiments, blocking LAG-3 is employed in the treatment of lung cancer.

In the context of the present specification, TIGIT refers to T cell immunoreceptor with Ig and immunoreceptor tyrosine-based inhibitory motif domains. In certain embodiments, blocking TIGIT is employed in the treatment of cancer.

In the context of the present specification, BTLA refers to B- and T-lymphocyte attenuator, also known as CD272. In certain embodiments, blocking BTLA is employed in the treatment of cancer.

In the context of the present specification, VISTA refers to V-domain Ig suppressor of T cell activation. In certain embodiments, blocking VISTA is employed in the treatment of cancer.

In the context of the present specification, CD96 refers to T cell activation, increased late expression, also known as TACTILE. In certain embodiments, blocking CD96 is employed in the treatment of cancer.

In the context of the present specification, 4-1BB refers to CD137, also known as TNFR superfamily member 9 or induced by lymphocyte activation or ILA. In certain embodiments, binding of 4-1BB is employed in the treatment of cancer.

In the context of the present specification, CCL-2 refers to chemokine (C-C motif) ligand 2 (CCL2), also known as monocyte chemoattractant protein 1 (MCP1) or small inducible cytokine A2. In certain embodiments, CCL-2 is employed in the treatment of cancer, stroke, and dementia. In certain embodiments, blocking of CCL-2 is employed in the treatment of autoimmune inflammation and cancer.

In the context of the present specification, IL-1 refers to members of the IL-1 cytokine family. In certain embodiments, blocking of IL-1 is employed in the treatment of multiple sclerosis.

In the context of the present specification, IL-1R refers to receptor for the cytokines of the IL-1 cytokine family. In certain embodiments, blocking of IL-1R is employed in the treatment of multiple sclerosis.

In the context of the present specification, EphA2 refers to ephrin type-A receptor 2. In certain embodiments, blocking EphA2 is employed in the treatment of cancer.

In the context of the present specification, EphA3 refers to ephrin type-A receptor 3. In certain embodiments, blocking EphA3 is employed in the treatment of cancer.

In the context of the present specification, EphB2 refers to ephrin type-B receptor 2, also known as ERK. In certain embodiments, blocking EphB2 is employed in the treatment of cancer.

In the context of the present specification, EphB3 refers to ephrin type-B receptor 3. In certain embodiments, blocking EphB3 is employed in the treatment of cancer.

In the context of the present specification, EphB4 refers to ephrin type-B receptor 4. In certain embodiments, blocking EphB4 is employed in the treatment of cancer.

In the context of the present specification, OX40 refers to TNFR superfamily member 4, also known as CD134 or OX40 receptor. In certain embodiments, binding OX40 is employed in the treatment of cancer.

In the context of the present specification, LINGO-1 refers to Leucine rich repeat and Immunoglobin-like domain-containing protein 1. In certain embodiments, blocking LINGO-1 is employed in the treatment of multiple sclerosis, traumatic brain CNS injury or stroke.

In the context of the present specification, L1CAM refers to L1 cell adhesion molecule, also known as L1. In certain embodiments, blocking L1 is employed in the treatment of multiple sclerosis, traumatic brain CNS injury or stroke.

In the context of the present specification, NCAM refers to neural cell adhesion molecule. In certain embodiments, blocking NCAM is employed in the treatment of multiple sclerosis, traumatic brain CNS injury or stroke.

In the context of the present specification, SOD-1 refers to superoxide dismutase 1. In certain embodiments, blocking SOD-1 is employed in the treatment of Amyotrophic Lateral Sclerosis (ALS).

In the context of the present specification, SIGMAR-1 refers to sigma-1 receptor. In certain embodiments, blocking SIGMAR-1 is employed in the treatment of Amyotrophic Lateral Sclerosis (ALS).

In the context of the present specification, SIGMAR-2 refers to sigma-2 receptor. In certain embodiments, blocking SIGMAR-2 is employed in the treatment of Amyotrophic Lateral Sclerosis (ALS).

In the context of the present specification, TDP-43 refers to TAR DNA-binding protein 43. In certain embodiments, binding TDP-43 is employed in the treatment of Amyotrophic Lateral Sclerosis (ALS).

In the context of the present specification, Aβ refers to amyloid beta. In certain embodiments, binding Aβ is employed in the treatment of Alzheimer's disease (AD).

In the context of the present specification, Tau refers to tau proteins. In certain embodiments, binding Tau is employed in the treatment of Alzheimer's disease (AD).

In the context of the present specification, IFNα refers to interferon-alpha. In certain embodiments, IFNα is employed in the treatment of cancer and infectious diseases.

In the context of the present specification, IFNβ refers to interferon-beta. In certain embodiments, IFN is employed in the treatment of cancer and infectious diseases.

In the context of the present specification, TRPM4 refers to Transient receptor potential cation channel subfamily M member 4. In certain embodiments, blocking TRPM4 is employed in the treatment of multiple sclerosis.

In the context of the present specification, AS/C1 refers to Acid-sensing ion channel 1, also known as amiloride-sensitive cation channel 2, neuronal (ACCN2) or brain sodium channel 2 (BNaC2). In certain embodiments, blocking ASIC1 is employed in the treatment of multiple sclerosis.

In the context of the present specification, VGCC refers to Voltage-gated calcium channels, also known as voltage-dependent calcium channels (VDCCs). In certain embodiments, blocking VGCC is employed in the treatment of multiple sclerosis.

In the context of the present specification, CB₁ refers to Cannabinoid receptor type 1, also known as cannabinoid receptor 1. In certain embodiments, blocking CB₁ is employed in the treatment of multiple sclerosis.

In the context of the present specification, TTR refers to Transthyretin. In certain embodiments, blocking TTR is employed in the treatment of transthyretin amyloidosis.

In the context of the present specification, HTT refers to huntingtin protein. In certain embodiments, blocking HTT is employed in the treatment of Huntington's disease.

In the context of the present specification, JCV refers to JC virus or John Cunningham virus. In certain embodiments, blocking major capsid protein VP1 (viral protein 1) of JCV is employed in the treatment of progressive multifocal leukoencephalopathy (PML).

In the context of the present specification, C9orf72 refers to the protein encoded by chromosome 9 open reading frame 72 gene. In certain embodiments, C9orf72 is employed in the treatment of dementia. In certain embodiments, blocking C9orf72 is employed in the treatment of dementia.

In the context of the present specification, BDNF refers to brain derived neurotrophic factor. In certain embodiments, BDNF is employed in the treatment of multiple sclerosis, Parkinson's disease, dementia, stroke and hereditary disorders.

In the context of the present specification, NRTN refers to neurturin. In certain embodiments, NRTN is employed in the treatment of multiple sclerosis, Parkinson's disease, dementia, stroke and hereditary disorders.

In the context of the present specification, ARTN refers to artemin. In certain embodiments, ARTN is employed in the treatment of multiple sclerosis, Parkinson's disease, dementia, stroke and hereditary disorders.

In the context of the present specification, PSPN refers to persephin. In certain embodiments, PSPN is employed in the treatment of multiple sclerosis, Parkinson's disease, dementia, stroke and hereditary disorders.

In the context of the present specification, CNTF refers to ciliary neurotrophic factor. In certain embodiments, CNTF is employed in the treatment of multiple sclerosis, Parkinson's disease, dementia, stroke and hereditary disorders.

In the context of the present specification, TRAIL refers to TNF-related apoptosis-inducing ligand, also known as CD253 or tumor necrosis factor superfamily, member 10. In certain embodiments, TRAIL is employed in the treatment of cancer.

In the context of the present specification, HA refers to hemagglutinin (or haemagglutinin), a homotrimeric glycoprotein found on the surface of influenza viruses. In certain embodiments, neutralizing HA is employed in the treatment of influenza.

In the context of the present specification, IL-3 refers to interleukin 3. In certain embodiments, IL-3 is employed in the treatment of cancer.

In the context of the present specification, IL-5 refers to interleukin 5. In certain embodiments, IL-5 is employed in the treatment of cancer. In certain embodiments, blocking of IL-5 is employed in the treatment of asthma. In certain embodiments, blocking of IL-5 is employed in the treatment of chronic obstructive pulmonary disease (COPD).

In the context of the present specification, IL-8 refers to interleukin 8, also known as chemokine (C-X-C motif) ligand 8 or CXCL8. In certain embodiments, IL-8 is employed in the treatment of cancer. In certain embodiments, blocking of IL-8 is employed in the treatment of lung oedema. In certain embodiments, an IL-8 antagonist is employed in the treatment of cystic fibrosis.

In the context of the present specification, IL-17 refers to interleukin 17. In certain embodiments, neutralisation of IL-17 is employed in the treatment of uveitis. In the context of the present specification, IL-17A refers to interleukin 17A. In certain embodiments, neutralisation of IL-17A is employed in the treatment of rheumatoid arthritis and/or psoriatic arthritis and/or ankylosing spondylitis.

In the context of the present specification, IL-18 refers to interleukin 18, also known as interferon-gamma inducing factor. In certain embodiments, IL-18 is employed in the treatment of cancer.

In the context of the present specification, IL-21 refers to interleukin 21. In certain embodiments, IL-21 is employed in the treatment of cancer.

In the context of the present specification, IL-21R refers to the interleukin 21 receptor. In certain embodiments, blocking of IL-21R is employed in the treatment of allergic asthma.

In the context of the present specification, IL-22 refers to interleukin 22. In certain embodiments, neutralising IL-22 is employed in the treatment of rheumatoid arthritis.

In the context of the present specification, IL-25 refers to interleukin 25 (also known as interleukin 17E, or IL-17E). In certain embodiments, neutralizing IL-25 is employed in the treatment of allergic asthma.

In the context of the present specification, CD20 refers to B-lymphocyte antigen CD20. In certain embodiments, CD20 binding antibodies are employed in the treatment of interstitial lung disease.

In certain embodiments CD20 binding antibodies are is employed for the treatment of cancer. In the context of the present specification, CCL5 refers to chemokine (C-C motif) ligand 5. In certain embodiments, CCL5 is employed in the treatment of cancer.

In the context of the present specification, CCL21 refers to chemokine (C-C motif) ligand 21. In certain embodiments, CCL21 is employed in the treatment of cancer.

In the context of the present specification, CCL10 refers to chemokine (C-C motif) ligand 10, also known as CCL9 or chemokine (C-C motif) ligand 9. In certain embodiments, CCL10 is employed in the treatment of cancer.

In the context of the present specification, CCL16 refers to chemokine (C-C motif) ligand 16.

In certain embodiments, CCL16 is employed in the treatment of cancer.

In the context of the present specification, CX3CL1 refers chemokine (C-X3-C motif) ligand 1, also known as fractalkine. In certain embodiments, CX3CL1 is employed in the treatment of cancer.

In the context of the present specification, CXCL16 refers to chemokine (C-X-C motif) ligand 16. In certain embodiments, CXCL16 is employed in the treatment of cancer.

In the context of the present specification, NF-k8 refers to nuclear factor kappa-light-chain-enhancer of activated B cells. In certain embodiments, an NF-kB antagonist is employed in the treatment of cystic fibrosis.

In the context of the present specification, NRA refers to non rheumatoid arthritis. In certain embodiments, anti-nerve growth factor (NGF) antibodies or antibody like molecules can be employed in the treatment of inflammation, autoimmune inflammation, arthritis and osteo arthritis. In certain embodiments, blocking of the NGF can be employed in the treatment of osteoarthritis. In the context of the present specification, the term antibody refers to antibodies of type G (IgG), any antigen binding fragment or single chains thereof and related or derived constructs. A whole antibody is a glycoprotein comprising at least two heavy (H) chains and two light (L) chains inter-connected by disulfide bonds. Each heavy chain is comprised of a heavy chain variable region (V_{H}) and a heavy chain constant region (C_{H}). The heavy chain constant region is comprised of three domains, C_{H}1, C_{H}2 and C_{H}3. Each light chain is comprised of a light chain variable region (abbreviated herein as V_{L}) and a light chain constant region (C_{L}). The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component of the classical complement system. In the context of the present specification, the term antibody is meant to include not only whole antibodies comprising two H chains and two L chains, but also unusual antibodies comprising only one H chain and one L chain, or even antibodies consisting of just one H chain.

The term specifically binding in the context of the present specification refers to binding with high affinity/a Kd≤10E⁻⁸ mol/l.

The term antibody-like molecule in the context of the present specification refers to a molecule containing at least a part of an Fc fragment of an IgG antibody and at least one target-binding element fused directly or indirectly to the Fc fragment, being heavy and light chain variable regions, single chain variable fragments, dual-affinity retargeting proteins or bispecific T cell engagers among others. Antibody-like molecule is capable of specific binding to another molecule or target with high affinity/a Kd≤10E⁻⁸ mol/l. An antibody-like molecule binds to its target similarly to the specific binding of an antibody.

The skilled person is aware that the current invention requires that the antibody or antibody-like molecule comprises an Fc region or is fused to an Fc region.

In the context of the present specification, the term dissociation constant (KD) refers to an equilibrium constant that measures the propensity of a complex composed of [mostly two] different components to dissociate reversibly into its constituent components. The complex can be e.g. an antibody-antigen complex AbAg composed of antibody Ab and antigen Ag. K_{D} is expressed in molar concentration [mol/l] and corresponds to the concentration of [Ab] at which half of the binding sites of [Ag] are occupied, in other words, the concentration of unbound [Ab] equals the concentration of the [AbAg] complex. The dissociation constant can be calculated according to the following formula:

$${K}_{D} = \frac{[ Ab ] * [ Ag ]}{[ AbAg ]}$$

[Ab]: concentration of antibody; [Ag]: concentration of antigen; [AbAg]: concentration of antibodyantigen complex

In the context of the present specification, the terms off-rate (Koff; [1/sec]) and on-rate (Kon; [1/sec*M]) are used in their meaning known in the art of chemistry and physics; they refer to a rate constant that measures the dissociation (Koff) or association (Kon) of 5 an antibody with its target antigen. K_{off} and Kₒₙ can be experimentally determined using methods well established in the art. A method for determining the Koff and Kon of an antibody employs surface plasmon resonance. This is the principle behind biosensor systems such as the Biacore® or the ProteOn® system. They can also be used to determine the dissociation constant K_{D} by using the following formula:

$${K}_{D} = \frac{[ {K}_{off} ]}{[ {K}_{on} ]}$$

In the context of the present specification, K_{D} can be also determined by equilibrium analysis of experimental data determined using methods well established in the art. This can be performed using biosensor systems such as the Biacore® or the ProteOn® system.

In the context of the present specification, high grade glioma (HGG) refers to a WHO grade IV glioma or glioblastoma multiforme.

In the context of the present specification, an Fc region with the designation “NHQ” refers to an Fc region in which the positions 253, 310 and 435 (as specified by the EU numbering system) comprise the indicated amino acid residues, in other words: N at position 253, H at position 310 and Q at position 435. This corresponds to an Fc region carrying two mutations: I253N and H435Q. Accordingly, an Fc region with the designation “IAQ” refers to an Fc region having I at position 253, A at position 310 and Q at position 435 (i.e. an Fc region carrying the mutations H310A and H435Q). Table 1 lists several examples of modified Fc regions.

A first aspect of the invention provides a fusion polypeptide comprising IL-12 and a crystallizable fragment (Fc) region of IgG, for use in prevention or treatment of a disease affecting the central nervous system. The Fc region bears a modification resulting in reduced affinity to the neonatal Fc receptor (FcRn). The polypeptide is administered to the brain.

Administration to the brain can be effected by intracranial delivery. Intracranial delivery may be continuous or intermittent or nonrecurring. The expression “administration to the brain” is also meant to include rinsing of a resection cavity following an operation. Administration may be intrathecal or intraparenchymal.

The modification of the Fc region results in a decreased serum to brain concentration ratio of the polypeptide. A decreased serum to brain concentration has the advantage that a high local concentration can be achieved within the brain, while negative side effects due to high systemic concentrations are prevented.

In certain embodiments, the serum or plasma to brain concentration ratio of the polypeptide is below a predetermined threshold. The predetermined threshold is selected from
a. at most ⅔ of the serum or plasma to brain concentration ratio of the same polypeptide comprising a non-modified Fc region, particularly IL-12Fc WT, or
b. at most ⅛ of the serum or plasma to brain concentration ratio of the same polypeptide neither comprising an Fc region nor peptide linkers, particularly rhIL-12
   measurable 24 h after intracranial injection, in particular intracranial bolus injection or CED, into the striatum of FcRn^{tg} mice.

The measurement is performed 24 h after intracranial injection into the striatum of FcRn^{tg} mice with 1 μl/min of 1 pg using a blunt end 26s G Hamilton syringe or CED (using a 27 G blunt-end needle with a 1 mm step at the tip made of fused silica with internal diameter of 0.1 mm and wall thickness of 0.0325 mm and a ramp-up injection regimen of 0.2 μl/minute for 5 minutes, 0.5 μl/minute for 4 minutes and 0.8 μl/minute for 2.5 minutes; total volume 5 μl, total amount 1 pg).

The fusion polypeptide according to the first aspect of the invention has a lower serum to brain concentration ratio than IL-12 linked to a non-modified Fc region (IL-12Fc WT). IL-12Fc WT has a long serum half-life live due to FcRn mediated recycling in the circulation.

The fusion polypeptide according to the first aspect of the invention has a lower serum to brain concentration ratio than rhIL-12, which shows high passive leakage from the brain.

In certain embodiments, the reduced affinity of said polypeptide to FcRn is characterized by a dissociation constant (K_{D}) selected from
a. a K_{D} that is at least 2× increased compared to a K_{D} characterizing binding of FcRn to the same polypeptide comprising a non-modified Fc region, and
b. a K_{D} that is at least 1.5× increased compared to a K_{D} characterizing binding of FcRn to the same polypeptide comprising a differently modified Fc region, namely one mutant selected from IAQ (bearing the mutations H310A and H435Q) and AAA (bearing the mutations I253A, H310A and H435A)

In certain embodiments, the K_{D} is at least 3× increased compared to a K_{D} characterizing binding of FcRn to the same polypeptide comprising a non-modified Fc region. In certain embodiments, the K_{D} is at least 4× increased compared to a K_{D} characterizing binding of FcRn to the same polypeptide comprising a non-modified Fc region. In certain embodiments, the K_{D} is at least 5× increased compared to a K_{D} characterizing binding of FcRn to the same polypeptide comprising a non-modified Fc region.

In certain embodiments, the K_{D} is at least 2× increased compared to a K_{D} characterizing binding of FcRn to the same polypeptide comprising said differently modified Fc region.

In certain embodiments, the differently modified Fc region is an Fc region having I at position 253, A at position 310 and Q at position 435 (IAQ).

In certain embodiments, the differently modified Fc region is an Fc region having A at position 253, A at position 310 and A at position 435 (AAA).

In certain embodiments, the intracranial delivery is effected by convection enhanced delivery (CED) or a variation thereof. CED refers to a technique that allows drugs to be delivered directly to the brain (-tumor) parenchyma. The CED procedure involves a minimally invasive surgical exposure of the brain, followed by placement of small diameter catheters directly into the brain, thereby bypassing the blood-brain-barrier. The main difference to regular bolus injection and diffusion driven infusion regimens is a pressure gradient that is created via ramping up the injection until bulk flow within the tissue is reached. Now the duration rather than the infusion rate determines the range of tissue reached. This approach allows for delivery of macromolecular drugs that would not normally enter the brain to effectively reach high concentrations within the brain (tumor) tissue.

In certain embodiments, the intracranial delivery is effected by intrathecal delivery. Intrathecal administration refers to direct administration of drugs into the cerebrospinal fluid (CSF). Intrathecal administration is defined as substance application below the subarachnoid membrane into the subarachnoid space in the brain (e.g. via the ommaya reservoir) or in the spinal cord. Non-limiting examples are intrathecal delivery to treat leptomeningeal carcinomatosis and primary Her2/neu positive brain tumors as well as CD20 positive CNS lymphoma and intraocular lymphoma, using trastuzumab or rituximab, respectively. Another example is intrathecal application of anti-NogoA antibodies for the treatment of acute spinal cord injury, multiple sclerosis or stroke. This approach allows for delivery of macromolecular drugs that would not normally enter the brain to effectively reach high concentrations at the leptomeninges or brain parenchyma.

In certain embodiments, the intracranial delivery is effected by intracerebroventricular delivery of said polypeptide. Intracerebroventricular administration refers to direct administration of drugs into the cerebrospinal fluid (CSF) by means of a cathether into the ventricular lumen.

In certain embodiments, the intracranial delivery is effected by in situ production of said polypeptide. In situ production relates to local production of the polypeptide exclusively or virtually exclusively within the brain or the brain tumor. By way of non-limiting example, local production may originate from DNA formulations, mRNA, modified mRNA, self-replicating mRNA, viral vectors, encapsulated modified producer cells or modified T cells. A spatial control over the local production can be achieved by local delivery of the molecules or vectors encoding the polypeptide or by local activation the production of the polypeptide. Local production via local delivery of the molecules or vectors encoding the polypeptide and subsequent local activation of the production of the polypeptide can be achieved via local or systemic administration of agents acting as transcriptional derepressors or transcriptional activators of conditional expression cassettes. Examples include but are not limited to ecdysone receptor/invertebrate retinoid x receptor-based inducible gene expression systems or tetracycline-regulated transcriptional modulators.

In certain embodiments, the intracranial delivery is effected by systemic delivery of cells modified to produce said polypeptide with homing capabilities to the tumor or CNS. The polypeptide may be produced in a constitutive or inducible manner. Examples include but are not limited to modified T cells or mesenchymal stem cells.

In certain embodiments, the intracranial delivery is effected by release from implanted slow-release/extended-release/sustained-release/controlled-release formulations. In the context of the present specification, such formulations relate to dosage forms designed to release a drug at a predetermined rate in order to maintain a constant drug concentration for a specific period of time with minimum side effects. The skilled person is aware of a variety of suitable formulations. Non-limiting examples are liposomes, drug-polymer conjugates, hydrogels, wavers or coated nanoparticles.

In certain embodiments, the intracranial delivery is effected by intranasal delivery of said polypeptide.

In certain embodiments, the intracranial delivery is effected by receptor mediated transcytosis of said polypeptide. A non-limiting example is a bispecific construct binding to TfR as well as a target found in the diseased brain parenchyma, particularly Aβ plaques in Alzheimer's Disease (AD).

In certain embodiments, the disease affecting the central nervous system is a malignant disease.

In certain embodiments, the disease affecting the central nervous system is a glioma.

In certain embodiments, the disease affecting the central nervous system is a high grade glioma (HGG).

In certain embodiments, the disease affecting the central nervous system a secondary brain tumor, also known as brain metastases.

In certain embodiments, the disease affecting the central nervous system is ischemic brain injury.

In certain embodiments, the disease affecting the central nervous system is cerebral infarction, stroke, brain hypoxia-ischemia, intracranial embolism or intracranial thrombosis.

In certain embodiments, the disease affecting the central nervous system is epilepsy.

In certain embodiments, the disease affecting the central nervous system is traumatic brain injury.

In certain embodiments, the disease affecting the central nervous system is a spinal cord injury.

In certain embodiments, the disease affecting the central nervous system is dementia.

In certain embodiments, the disease affecting the central nervous system is Parkinson's Disease (PD).

In certain embodiments, the disease affecting the central nervous system is dementia with Lewy Bodies.

In certain embodiments, the disease affecting the central nervous system is Alzheimer's Disease (AD). In certain embodiments, the disease affecting the central nervous system is familial Alzheimer's Disease (AD).

In certain embodiments, the disease affecting the central nervous system is frontotemporal dementia (FTD).

In certain embodiments, the disease affecting the central nervous system is familial frontotemporal dementia (FTD).

In certain embodiments, the disease affecting the central nervous system is Amyotrophic Lateral Sclerosis (ALS), also known as Lou Gehrig's Disease.

In certain embodiments, the disease affecting the central nervous system is a transmissible spongiform encephalopathy, particularly Creutzfeld Jakob Disease (CJD), Kuru, Scrapie, Bovine spongiform encephalopathy (BSE).

In certain embodiments, the disease affecting the central nervous system is a hereditary disorder.

In certain embodiments, the disease affecting the central nervous system is a hereditary disorder, particularly Cerebral Autosomal Dominant Arteriopathy with Subcortical Infarcts and Leukoencephalopathy (CADASIL)

In certain embodiments, the disease affecting the central nervous system is a hereditary disorder, particularly Huntington's Disease.

In certain embodiments, the disease affecting the central nervous system is a hereditary disorder, particularly Autism, autism spectrum disorders (ASD), e.g. Asperger Syndrome.

In certain embodiments, the disease affecting the central nervous system is hereditary Leukodystrophy, particularly metachromatic leukodystrophy, Krabbe Disease, Canavan Disease, X-linked Adrenoleukodystrophy, Alexander Disease.

In certain embodiments, the disease affecting the central nervous system is a hereditary metabolic disorder, particularly Tay-Sachs Disease or Wilson Disease.

In certain embodiments, the disease affecting the central nervous system is a psychiatric disorder, particularly amnesia, attention-deficit hyperactivity disorder, psychosis, anxiety disorders, bipolar disorders, depression, mania, intellectual developmental disorder, global developmental delay, post-traumatic stress disorder, acute stress disorder, dissociative disorders.

In certain embodiments, the disease affecting the central nervous system is epilepsy.

In certain embodiments, the disease affecting the central nervous system is autoimmune encephalitis.

In certain embodiments, the disease affecting the central nervous system is multiple sclerosis.

In certain embodiments, the disease affecting the central nervous system is neuromyelitis optica (NMO).

In certain embodiments, the disease affecting the central nervous system is autoimmune encephalitis, particularly anti-NMDAR encephalitis, limbic encephalitis, LGI1/CASPR2-antibody encephalitis, hashimoto's encephalopathy, acute Disseminated Encephalomyelitis (ADEM), Binswanger's Disease (Subcortical Leukoencephalopathy), Rasmussen's Encephalitis.

In certain embodiments, the disease affecting the central nervous system is infectious encephalomyelitis caused by viruses, particularly rabies virus, human herpes viruses, rash-causing viruses, insect-borne viruses, tick-borne viruses, human immunodeficiency virus (HIV).

In certain embodiments, the disease affecting the central nervous system is infectious encephalomyelitis caused by bacteria.

In certain embodiments, the disease affecting the central nervous system is infectious encephalomyelitis caused by parasites.

In certain embodiments, the disease affecting the central nervous system is progressive multifocal leukoencephalopathy (PML) caused by JC polyomavirus (usually abbreviated as JCPyV or JCV)

In certain embodiments, the disease affecting the central nervous system is postinfectious encephalomyelitis.

In certain embodiments, the disease affecting the central nervous system is neovascular age-related macular degeneration (wet AMD) and diabetic macular edema or retinitis pigmentosa.

In a further aspect of the invention, the polypeptide according to the invention is used for prevention or treatment of a disease affecting the lung, said disease being selected from coronavirus disease 2019, severe acute respiratory syndrome, asthma, allergic asthma, severe uncontrolled asthma, fibrosis, cystic fibrosis, pulmonary fibrosis, chronic obstructive pulmonary disease, influenza, lung oedema, sarcoidosis, lung cancer, tuberculosis, human orthopneumovirus, bubonic plague, pneumonic plague, anthrax, invasive fungal disease in lung, pulmonary paracoccidioidomycosis, interstitial lung disease, idiopathic pulmonary fibrosis, and chronic rhinosinusitis with nasal polyps.

In certain embodiments, the disease affecting the lungs is coronavirus disease 2019 (COVID-19) caused by severe acute respiratory syndrome coronavirus 2 (SARS-CoV-2).

In certain embodiments, the disease affecting the lungs is severe acute respiratory syndrome (SARS).

In certain embodiments, the disease affecting the lungs is severe acute respiratory syndrome (SARS) caused by a virus, in particular a coronavirus.

In certain embodiments, the disease affecting the lungs is asthma, allergic asthma, severe uncontrolled asthma, or a combination thereof.

In certain embodiments, the disease affecting the lungs is chronic obstructive pulmonary disease (COPD).

In certain embodiments, the disease affecting the lungs is fibrosis, cystic fibrosis, pulmonary fibrosis, or a combination thereof.

In certain embodiments, the disease affecting the lungs is influenza caused by an influenza virus.

In certain embodiments, the disease affecting the lungs is sarcoidosis (also known as Besnier-Boeck-Schaumann disease).

In certain embodiments, the disease affecting the lungs is lung cancer.

In certain embodiments, in general terms, the disease affecting the lungs is caused by a virus, bacterium, fungus or parasite.

In certain embodiments, the disease affecting the lungs is tuberculosis caused by Mycobacterium tuberculosis (usually abbreviated as M. tuberculosis or M. tb).

In certain embodiments, the disease affecting the lungs is respiratory tract infections caused by the syncytial virus human orthopneumovirus (also known as human respiratory syncytial virus, or HRSV, or just RSV).

In certain embodiments, the disease affecting the lungs is bubonic plague caused by bacterium Yersinia pestis.

In certain embodiments, the disease affecting the lungs is pneumonic plague caused by the bacterium Yersinia pestis.

In certain embodiments, the disease affecting the lungs is anthrax, an infection caused by the bacterium Bacillus anthracis.

In certain embodiments, the disease affecting the lungs is invasive fungal disease (also known as fungal lung disease) caused by pulmonary fungal pathogens such as Aspergillus, Cryptococcus, Pneumocystis, and endemic fungi.

In certain embodiments, the disease affecting the lungs is pulmonary paracoccidioidomycosis (typically abbreviated as PCM) caused by the fungus Paracoccidioides brasiliensis.

In certain embodiments, the disease affecting the lungs is chronic rhinosinusitis with nasal polyps (typically abbreviated as CRSwNP), a subgroup of chronic rhinosinusitis (CRS).

In certain embodiments, the disease affecting the lungs is lung oedema.

In certain embodiments, the disease affecting the lungs is interstitial lung disease.

In certain embodiments, the disease affecting the lungs is idiopathic pulmonary fibrosis.

In a further aspect of the invention, the polypeptide according to the invention is used for prevention or treatment of a disease affecting at least one joint, said disease being selected from rheumatoid arthritis, juvenile rheumatoid arthritis, gout, pseudogout, osteoarthritis, chronic hemophilic synovitis, psoriatic arthritis, and ankylosing spondylitis.

In certain embodiments, the disease affecting a joint is rheumatoid arthritis (RA). In certain embodiments, the disease affecting a joint is juvenile rheumatoid arthritis.

In certain embodiments, the disease affecting a joint is gout, a form of inflammatory arthritis caused by persistently elevated levels of uric acid in the blood. In certain embodiments, the disease affecting a joint is pseudogout.

In certain embodiments, the disease affecting a joint is osteoarthritis (OA) resulting from breakdown of joint cartilage and underlying bone.

In certain embodiments, the disease affecting a joint is chronic hemophilic synovitis.

In certain embodiments, the disease affecting a joint is psoriatic arthritis, a long-term inflammatory arthritis that occurs in people affected by the autoimmune disease psoriasis.

In certain embodiments, the disease affecting a joint is ankylosing spondylitis (also known as Bekhterev's disease, Bechterew's disease, or morbus Bechterew).

In a further aspect of the invention, the polypeptide according to the invention is used for prevention or treatment of a disease affecting the eye, said disease being selected from uveal melanoma and uveitis.

In certain embodiments, the disease affecting the eye is uveal melanoma, a cancer (melanoma) of the eye involving the iris, ciliary body, or choroid (collectively referred to as the uvea).

In certain embodiments, the disease affecting the eye is uveitis, i.e. the inflammation of the uvea.

It is understood that the polypeptide according to the invention can be used for prevention or treatment of multiple diseases or a combination of diseases disclosed herein simultaneously and/or successively. In certain embodiments, the Fc region is a chimeric Fc region comprising a human or humanized amino acid sequence.

In certain embodiments, the Fc region is a human or humanized Fc region.

In certain embodiments, the Fc region bears a mutation at position 253 relative to SEQ ID NO 1.

In certain embodiments, the Fc region bears the mutation I253A. In certain embodiments, the Fc region bears the mutation I253N.

In certain embodiments, the Fc region bears a mutation at position 435 relative to SEQ ID NO 1.

In certain embodiments, the Fc region bears the mutation H435Q.

In certain embodiments, the Fc region does not bear a mutation at position 435 relative to SEQ ID NO 1. Thus, the Fc region bears a H at position 435.

In certain embodiments, the Fc region does not bear a mutation at position 310 relative to SEQ ID NO 1. Thus, the Fc region bears a H at position 310.

In certain embodiments, the Fc region comprises
the mutations I253A and H435Q, and an H at position 310 (AHQ);
the mutations I253N and H435Q, and an H at position 310 (NHQ);
the mutations I253A, H310A and H435Q (AAQ);
the mutations I253N, H310A and H435Q (NAQ);
the mutation I253A and an H at position 310 and 435 (AHH);
the mutation I253N and an H at position 310 and 435 (NHH);
the mutations I253A and H310A, and an H at position 435 (AAH);
the mutations I253N and H310A, and an H at position 435 (NAH);
the mutations I253N, H310A and H435A (NAA);
the mutations I253N, H310A and H435E (NAE);
the mutations I253A, H310A and H435A (AAA); or
the mutations I253A, H310A and H435E (AAE).

In certain embodiments, the Fc region comprises
the mutations I253N and H435Q, and an H at position 310 (NHQ);
the mutations I253A, H310A and H435Q (AAQ);
the mutations I253N, H310A and H435Q (NAQ);
the mutations I253N, H310A and H435E (NAE); or
the mutations I253A, H310A and H435E (AAE).

In certain embodiments, the Fc region comprises the mutations I253N and H435Q, and an H at position 310.

In certain embodiments, the Fc region comprises the mutations I253A, H310A and H435Q (AAQ).

In certain embodiments, the Fc region comprises the mutations I253N, H310A and H435Q (NAQ).

In certain embodiments, the Fc region comprises the mutations I253N, H310A and H435E (NAE).

In certain embodiments, the Fc region comprises the mutations I253A, H310A and H435E (AAE).

In certain embodiments, the Fc region is or comprises a sequence characterized by SEQ ID NO 002 (IAQ), SEQ ID NO 003 (AHQ), SEQ ID NO 004 (NHQ), SEQ ID NO 005 (AAQ), SEQ ID NO 006 (NAQ), SEQ ID NO 007 (AHH), SEQ ID NO 008 (NHH), SEQ ID NO 009 (AAH), SEQ ID NO 010 (NAH), SEQ ID NO 011 (NAA), SEQ ID NO 012 (NAE), SEQ ID NO 013 (AAA) or SEQ ID NO 014 (AAE).

In certain embodiments, the Fc region is or comprises a sequence characterized by SEQ ID NO 004 (NHQ), SEQ ID NO 005 (AAQ), SEQ ID NO 006 (NAQ), SEQ ID NO 012 (NAE) or SEQ ID NO 014 (AAE).

In certain embodiments, the Fc region is or comprises a sequence characterized by SEQ ID NO 004 (NHQ).

In certain embodiments, the Fc region is or comprises a sequence characterized by SEQ ID NO 006 (NAQ).

In certain embodiments, the Fc region is or comprises a sequence characterized by SEQ ID NO 012 (NAE).

In certain embodiments, the Fc region is or comprises a sequence characterized by SEQ ID NO 014 (AAE).

Polypeptide comprising a crystallizable fragment (Fc) region for use in treatment A broader aspect of the invention provides a polypeptide comprising a crystallizable fragment (Fc) region of IgG, for use in prevention or treatment of a disease. The Fc region bears a modification resulting in reduced affinity to the neonatal Fc receptor (FcRn), and the polypeptide is delivered by local administration to the tissue affected by the disease.

In certain embodiments, the polypeptide is delivered to the eye by intraocular administration.

In certain embodiments, the polypeptide is delivered to a joint by intraarticular administration.

In certain embodiments, the polypeptide is delivered to the lungs via inhalation.

The invention further provides a polypeptide comprising a crystallisable fragment (Fc) region of IgG, preferably further comprising
IL-12; or
a polypeptide binding to any one of VEGFR, Ang2, TNFα, IL-17, PD-1, PD-L1, more preferably a polypeptide binding to any one of VEGFR, Ang2, TNFα, IL-17;
   for use in prevention or treatment of a disease affecting the eye, in particular a neoplastic disease affecting the eye, wherein said Fc region bears a modification resulting in reduced affinity to the neonatal Fc receptor (FcRn), said Fc comprises the mutations I253N and H435Q and an H at position 310 (NHQ) and said polypeptide is delivered to the eye by intraocular administration.

The invention further provides a polypeptide comprising a crystallisable fragment (Fc) region of IgG, preferably further comprising
IL-12; or
a polypeptide binding to any one of TNFα, IL-1 RA, IL-6R, IL-6, CD27, IL-22, IL-17, CD27, more preferably a polypeptide binding any one of TNFα, IL-1RA, IL-6R, IL-6, CD27;
   for use in prevention or treatment of a disease affecting a joint, wherein said Fc region bears a modification resulting in reduced affinity to the neonatal Fc receptor (FcRn), said Fc comprises the mutations I253N and H435Q and an H at position 310 and said polypeptide is delivered to said joint by intraarticular administration.

The invention further provides a polypeptide comprising a crystallisable fragment (Fc) region of IgG, preferably further comprising
IL-12; or
IL-10; or
a polypeptide binding to any one of IL-4RA, TNFα, IL-5, IL-6R, PD-1, PD-L1, CTLA-4, IL-8, IL-21R, CD25, CD20, NF-kB; more preferably a polypeptide binding to any one of IL-4RA, TNFα, IL-5, IL-6R, PD-1, PD-L1, CTLA-4;
   for use in prevention or treatment of a disease affecting the lungs, wherein said Fc region bears a modification resulting in reduced affinity to the neonatal Fc receptor (FcRn), said Fc comprises the mutations I253N and H435Q and an H at position 310 and said polypeptide is delivered to the lungs via inhalation.

The invention further provides a fusion polypeptide comprising a crystallisable fragment (Fc) region of IgG, in particular further comprising IL-12, wherein said Fc region bears a modification resulting in reduced affinity to the neonatal Fc receptor (FcRn), said Fc comprises the mutations I253N and H435Q and an H at position 310, for use as a medicament.

In certain embodiments, the crystallisable fragment (Fc) region of the polypeptide for use in prevention or treatment of a disease is or comprises a sequence SEQ ID NO 004 (NHQ). In certain embodiments, the crystallisable fragment (Fc) region of the fusion polypeptide for use as a medicament is or comprises a sequence SEQ ID NO 004 (NHQ).

Following local administration, the reduced affinity to FcRn ensures that transport into the circulation and systemic enrichment is reduced, thereby reducing any systemic toxic side effects of the polypeptide.

In certain embodiments, the polypeptide is selected from
a. a fusion protein comprising
   i. an effector polypeptide and
   ii. said Fc region; or
b. an antibody or antibody-like molecule comprising or linked to said Fc region.

Further embodiments of the polypeptide comprising a crystallizable fragment (Fc) region for use in treatment can be found in the “items” section below.

Targeting the PD-1/PD-L1 Axis for Use in Treatment

Another aspect of the invention provides an antibody or antibody-like molecule specifically binding to programmed cell death protein 1 (PD-1) or programmed death-ligand 1 (PD-L1) for use in prevention or treatment of a disease affecting the central nervous system. The antibody or antibody-like molecule comprises an Fc region bearing a modification resulting in reduced affinity to the neonatal Fc receptor (FcRn). The antibody or antibody-like molecule is administered to the central nervous system, in particular the brain.

Anti-OX40 for Use in Treatment

Another aspect of the invention provides an antibody or antibody-like molecule specifically binding to tumor necrosis factor receptor superfamily, member 4 (TNFRSF4), also known as CD134, OX40 or OX40 receptor for use in prevention or treatment of a disease affecting the central nervous system. The antibody or antibody-like molecule comprises an Fc region bearing a modification resulting in reduced affinity to the neonatal Fc receptor (FcRn). The antibody or antibody-like molecule is administered to the brain.

Anti-CD47 for Use in Treatment

Another aspect of the invention provides an antibody or antibody-like molecule specifically binding to CD47, also known as integrin associated protein (IAP) for use in prevention or treatment of a disease affecting the central nervous system. Yet another aspect of the invention provides a ligand of CD47, particularly SIRPα or thrombospondin-1 (TSP-1) fused with an Fc region. The antibody or antibody-like molecule or Fc-fusion molecule comprises an Fc region bearing a modification resulting in reduced affinity to the neonatal Fc receptor (FcRn). The antibody or antibody-like molecule or Fc-fusion molecule is administered to the brain.

Anti-Nogo-A for Use in Treatment

Another aspect of the invention provides an antibody or antibody-like molecule specifically binding to Nogo-A for use in prevention or treatment of a disease affecting the central nervous system. Yet another aspect of the invention provides a ligand of Nogo-A, particularly the Nogo-66 Receptor also known as Nogo Receptor 1 (NgR1) fused with an Fc region. The antibody or antibody-like molecule or Fc-fusion molecule comprises an Fc region bearing a modification resulting in reduced affinity to the neonatal Fc receptor (FcRn). The antibody or antibody-like molecule or Fc-fusion molecule is administered to the brain.

T Cell Engaging Bispecific Antibodies for Use in Treatment

Another aspect of the invention provides T cell engaging bispecific antibodies, bispecific antibodies or antibody-like molecules specifically binding to a tumor associated antigen (TAA) on cancer cells and at the same time to CD3 on T cells providing T cell receptor independent polyclonal activation if bound to the TAA for use in prevention or treatment of a disease affecting the central nervous system. Yet another aspect of the invention provides a bispecific antibody or antibody-like molecule specifically binding to PD-L1 and at the same time to 4-1BB on T cells. An even further aspect of the invention provides a bispecific antibody or antibody-like molecule specifically binding to PD-L1 and at the same time to CD28 on T cells. The antibody or antibody-like molecule or Fc-fusion molecule comprises an Fc region bearing a modification resulting in reduced affinity to the neonatal Fc receptor (FcRn). The antibody or antibody-like molecule or Fc-fusion molecule is administered to the brain.

Armed Antibodies and Targeted Antibodies for Use in Treatment

Another aspect of the invention provides an armed antibody or antibody-like molecule specifically binding to a tumor associated antigen (TAA) on cancer cells or to an antigen present in the tumor vasculature or an antigen present in the necrotic core of the tumor. Said antibody or antibody-like molecule also carries an effector molecule, particularly a cytokine, a radioactive isotope or a cytotoxic substance for use in prevention or treatment of a disease affecting the central nervous system. The armed antibody or antibody-like molecule or Fc-fusion molecule comprises an Fc region bearing a modification resulting in reduced affinity to the neonatal Fc receptor (FcRn). The antibody or antibody-like molecule or Fc-fusion molecule is administered to the brain.

Tumor Conditional or Tissue Conditional Antibodies

Another aspect of the invention provides an antibody or antibody-like molecule that binds specifically to a first tumor associated antigen (TAA) on cancer cells or to an antigen present in the tumor microenvironment or an antigen present in the necrotic core of the tumor or an antigen present in the target tissue. Said antibody or antibody-like molecule also comprises a second effector molecule, particularly a cytokine or is a bispecific or multispecific antibody with at least one more antibody or antibody-like molecule binding a second antigen different from the first for use in prevention or treatment of a disease affecting the central nervous system. In such a construct, the second antibody or antibody-like domain is shielded and cannot bind its target antigen. The shielding domain is connected to the second antibody or antibody-like construct via a protease sensitive linker peptide that will be cleaved in the tumor or target tissue by proteases predominantly or exclusively found there. The shielding domain can be the antibody or antibody-like molecule binding the first antigen. Upon cleavage of the shielding domain in the target tissue or tumor microenvironment the second antibody or antibody-like molecule will bind its target antigen or, in case of a cytokine or chemokine, will bind to its receptor. The tumor conditional or tissue conditional antibody or antibody-like molecule or Fc-fusion molecule comprises an Fc region bearing a modification resulting in reduced affinity to the neonatal Fc receptor (FcRn). The antibody or antibody-like molecule or Fc-fusion molecule is administered to the brain.

The skilled person is aware that in the case of an antibody, the antibody itself already comprises an Fc region. In the case of an antibody-like molecule, the antibody-like molecule or Fc-fusion molecule is linked to an Fc region.

Further embodiments of the antibody or antibody-like molecule or Fc-fusion molecule comprising a crystallizable fragment (Fc) region for use in treatment can be found in the “items” section below.

Polypeptide Comprising Fc Region with a Reduced Affinity to FcRn (Increased K_{D})

A second aspect of the invention provides a polypeptide comprising a Fc region of IgG, wherein said Fc region bears a modification resulting in reduced affinity to the neonatal Fc receptor (FcRn) compared to the affinity of the same polypeptide comprising a non-modified Fc region.

In certain embodiments, the reduced affinity of said polypeptide to FcRn is characterized by a dissociation constant (K_{D}) selected from
a. a K_{D} that is at least 2× increased compared to a K_{D} characterizing binding of FcRn to the same polypeptide comprising a non-modified Fc region, and
b. a K_{D} that is at least 1.5× increased compared to a K_{D} characterizing binding of FcRn to the same polypeptide comprising a differently modified Fc region, namely one mutant selected from IAQ (bearing the mutations H310A and H435Q) and AAA (bearing the mutations I253A, H310A and H435A)

In certain embodiments, the K_{D} is at least 3× increased compared to a K_{D} characterizing binding of FcRn to the same polypeptide comprising a non-modified Fc region. In certain embodiments, the K_{D} is at least 4× increased compared to a K_{D} characterizing binding of FcRn to the same polypeptide comprising a non-modified Fc region. In certain embodiments, the K_{D} is at least 5× increased compared to a K_{D} characterizing binding of FcRn to the same polypeptide comprising a non-modified Fc region.

In certain embodiments, the K_{D} is at least 2× increased compared to a K_{D} characterizing binding of FcRn to the same polypeptide comprising said differently modified Fc region.

In certain embodiments, the polypeptide is selected from
a. a fusion protein comprising
   i. an effector polypeptide and
   ii. said Fc region; or
b. an antibody or antibody-like molecule comprising or linked to said Fc region.

The skilled person is aware that in the case of an antibody, the antibody itself already comprises an Fc region. In the case of an antibody-like molecule, the antibody-like molecule is linked to an Fc region.

In certain embodiments, the effector polypeptide has the function of
a. a cytokine or hormone or growth factor,
b. a cytokine receptor or hormone receptor or growth factor receptor, or
c. a metabolite
   and is known to have a therapeutic or preventive effect on a disease, in particular on a disease affecting the central nervous system.

In certain embodiments, the effector polypeptide is able to specifically bind to the extracellular matrix (ECM) and is known to have a therapeutic or preventive effect on a disease, in particular on a disease affecting the central nervous system. In certain embodiments, the effector polypeptide is able to specifically bind to RNA and is known to have a therapeutic or preventive effect on a disease, in particular on a disease affecting the central nervous system.

In certain embodiments, the effector polypeptide is selected from the group comprising IL-12, IL-10, IL-2, IL-7, IFNα, IFNβ, IFNγ, IL-15, TNFα, CTLA-4, TGFβ, TGFβRII, GDNF, IL-35, CD95, IL-1RA, IL-4, IL-13, IL-33, IL-23, SIRPα, G-CSF, GM-CSF, OX40L, CD80, CD86, GITRL, 4-1BBL, EphrinA1, EphrinB2, EphrinB5, BDNF, C9orf72, NRTN, ARTN, PSPN, CNTF, TRAIL, IL-4, IL-3, IL-1, IL-5, IL-8, IL-18, IL-21, CCL5, CCL21, CCL10, CCL16, CX3CL1, CXCL16 in particular said effector polypeptide is IL-12.

In certain embodiments, the antibody or antibody-like molecule is selected from an antibody or antibody-like molecule specifically binding to PD-L1, TNFα, Histone, IFNγ, CXCL10, CTLA4, PD-1, CD3, OX40, CD20, CD22, CD25, CD28, TREM2, IL-6, CX3CR1, Nogo-A, CD27, IL-12, IL-12Rb1, IL-23, IL-17, CD47, TGFR, EGFR, EGFRvIII, Her2, PDGFR, TGFR, FGFR, IL-4RA, TfR, LfR, IR, LDL-R, LRP-1, CD133, CD111, VEGFR, VEGF-A, Ang-2, IL-10, IL-10R, IL-13Rα2, α-synuclein, CSF1R, G-CSF, GM-CSF, GITR, TIM-3, LAG-3, TIGIT, BTLA, VISTA, CD96, CD147, 4-1BB, CCL2, IL-1 or IL-1R, EphA2, EphA3, EphB2, EphB3, EphB4, LINGO-1, L1CAM, NCAM, SOD-1, SIGMAR-1, SIGMAR-2, TDP-43, Aβ, Tau, IFNα, IFNβ, TRPM4, ASIC1, VGCCs, CB₁, TTR, HTT, JCV, C9orf72 in an agonistic or antagonistic fashion.

An antibody or antibody-like molecule according to the above aspect of the invention may be an antibody-like molecule derived from the recognition site of a physiological ligand of PD-1 or PD-L1 or PD-L2 or a full antibody. Such antibody or antibody-like molecule competes with the physiological ligand for binding to PD-1 or PD-L1 or PD-L2, respectively. Particularly, a non-agonist PD-1 antibody or antibody-like molecule or non-agonist PD-L1 antibody or antibody-like molecule or non-agonist PD-L2 antibody or antibody-like molecule does not lead to attenuated T cell activity when binding to PD-1, on the surface on a T-cell.

In some embodiments, non-agonist PD-1 antibodies or antibody-like molecules used in the present invention are able, when bound to PD-1, to sterically block interaction of PD-1 with its binding partners PD-L1 and/or PD-L2.

In some embodiments, said non-agonist PD-1 antibody or antibody-like molecule is a gamma immunoglobulin binding to PD-1, without triggering the physiological response of PD-1 interaction with its binding partners PD-L1 and/or PD-L2.

In some embodiments, said non-agonist PD-L1 (PD-L2) antibody or antibody-like molecule is a gamma immunoglobulin binding to PD-L1 (PD-L2), without triggering the physiological response of PD-1 interaction with its binding partners PD-L1 and/or PD-L2.

Non-limiting examples for a PD-1 antibody are the clinically approved antibodies pembrolizumab (CAS No. 1374853-91-4) and nivolumab (CAS No. Number 946414-94-4)

Non-limiting examples for a PD-L1 antibody are the clinically approved antibodies atezolizumab (CAS No. 1380723-44-3), durvalumab (CAS No. 1428935-60-7) and avelumab (CAS No. 1537032-82-8).

Non-limiting examples for a PD-1/PD-L1 or PD-L2 antibody currently undergoing clinical development are the antibodies MDX-1105/BMS-936559 or AMP-224. A non-limiting example of an antibody specifically binding to IL-12/23 is ustekinumab (CAS No. 815610-63-0).

In certain embodiments, the antibody or antibody-like molecule is an antibody specifically binding to PD-L1.

In some embodiments, agonistic OX40 antibodies or antibody-like molecules used in the present invention are able to trigger a signalling cascade in OX40 expressing cells upon binding to OX40 and in the absence of OX40 ligand.

Non-limiting examples for an OX40 antibody are the antibodies PF-04518600/PF-8600m BMS-986178, GSK3174998, MOXR0916, INCAGN01949, tavolimab/MEDI0562, currently undergoing clinical development.

In certain embodiments, the antibody or antibody-like molecule is an antibody specifically binding to OX40.

In some embodiments, antibodies or antibody-like molecules used in the present invention are able to block the interaction between CD47 and SIRPα signals which prevent phagocytosis of cancer cells.

Non-limiting examples of CD47 blocking antibodies or SIRPα fusion proteins are Hu5F9-G4, CC-90002/INBRX-103, IBI188, OSE-172, NI-1801, DSP107, TTI-622, TTI-621, ALX148, and SRF231.

In certain embodiments, the antibody or antibody-like molecule is an antibody specifically binding to Nogo-A.

In certain embodiments, the antibody or antibody-like molecule is a bispecific construct able to bind two antigens at the same time.

In certain embodiments, the antibody or antibody-like molecule is a trispecific construct.

In certain embodiments, the antibody or antibody-like molecule is a multispecific construct.

In certain embodiments, the antibody or antibody-like molecule is an antibody directed against histones present in the necrotic core of tumors, which is armed with IL-12 or IL-2. In certain instances armed antibodies are immunocytokines. Non-limiting examples of armed antibodies as immunocytokines are NHS-IL-12, NHS-IL2LT, Hu14.18-IL2, HuKS-IL2, huBC1-IL-12.

In certain embodiments, the Fc region is a chimeric Fc region comprising a human amino acid sequence.

In certain embodiments, the Fc region is a human Fc region.

In certain embodiments, the Fc region bears a mutation at position 253. In certain embodiments, the Fc region bears the mutation I253A. In certain embodiments, the Fc region bears the mutation I253N.

In certain embodiments, the Fc region bears a mutation at position 435. In certain embodiments, the Fc region bears the mutation H435Q.

In certain embodiments, the Fc region does not bear a mutation at position 435. Thus, the Fc region bears a H at position 435.

In certain embodiments, the Fc region does not bear a mutation at position 310. Thus, the Fc region bears a H at position 310.

In certain embodiments, the Fc region comprises
the mutations I253A and H435Q, and an H at position 310 (AHQ);
the mutations I253N and H435Q, and an H at position 310 (NHQ);
the mutations I253A, H310A and H435Q (AAQ);
the mutations I253N, H310A and H435Q (NAQ);
the mutation I253A and an H at position 310 and 435 (AHH);
the mutation I253N and an H at position 310 and 435 (NHH);
the mutations I253A and H310A, and an H at position 435 (AAH);
the mutations I253N and H310A, and an H at position 435 (NAH);
the mutations I253N, H310A and H435A (NAA);
the mutations I253N, H310A and H435E (NAE);
the mutations I253A, H310A and H435A (AAA); or
the mutations I253A, H310A and H435E (AAE).

In certain embodiments, the Fc region comprises
the mutations I253N and H435Q, and an H at position 310 (NHQ);
the mutations I253A, H310A and H435Q (AAQ);
the mutations I253N, H310A and H435Q (NAQ);
the mutations I253N, H310A and H435E (NAE); or
the mutations I253A, H310A and H435E (AAE).

In certain embodiments, the Fc region comprises the mutations I253N and H435Q, and an H at position 310.

In certain embodiments, the Fc region comprises the mutations I253A, H310A and H435Q (AAQ).

In certain embodiments, the Fc region comprises the mutations I253N, H310A and H435Q (NAQ).

In certain embodiments, the Fc region comprises the mutations I253N, H310A and H435E (NAE).

In certain embodiments, the Fc region comprises the mutations I253A, H310A and H435E (AAE).

In certain embodiments, the Fc region is or comprises a sequence characterized by SEQ ID NO 002 (IAQ), SEQ ID NO 003 (AHQ), SEQ ID NO 004 (NHQ), SEQ ID NO 005 (AAQ), SEQ ID NO 006 (NAQ), SEQ ID NO 007 (AHH), SEQ ID NO 008 (NHH), SEQ ID NO 009 (AAH), SEQ ID NO 010 (NAH), SEQ ID NO 011 (NAA), SEQ ID NO 012 (NAE), SEQ ID NO 013 (AAA) or SEQ ID NO 014 (AAE).

In certain embodiments, the Fc region is or comprises a sequence characterized by SEQ ID NO 004 (NHQ), SEQ ID NO 005 (AAQ), SEQ ID NO 006 (NAQ), SEQ ID NO 012 (NAE) or SEQ ID NO 014 (AAE).

In certain embodiments, the Fc region is or comprises a sequence characterized by SEQ ID NO 004 (NHQ).

In certain embodiments, the Fc region is or comprises a sequence characterized by SEQ ID NO 006 (NAQ).

In certain embodiments, the Fc region is or comprises a sequence characterized by SEQ ID NO 012 (NAE).

In certain embodiments, the Fc region is or comprises a sequence characterized by SEQ ID NO 014 (AAE).

Nucleic Acid

Another aspect of the invention provides a nucleic acid encoding the polypeptide according to the above aspect of the invention.

Virus

Another aspect of the invention provides a viral vector comprising the nucleic acid according to the above aspect of the invention. The viral vector can be a replicating or non-replicating virus suitable for application to a patient in treatment.

In certain embodiments of any aspect of the invention, the polypeptide comprising a modified Fc region according to the invention is used in combination with an FcRn-blocking antibody. FcRn-blocking antibodies are capable of inhibiting the binding between Fc-comprising polypeptides and FcRn, thus mimicking the technical effect of the invention. The combination with an FcRn-blocking antibody may enhance the described advantages of a polypeptide comprising a modified Fc region according to the invention.

In certain embodiments of any aspect of the invention, the Fc region is an Fc region of immunoglobulin G (IgG). IgG is a major effector molecule of the humoral immune response in man. There are four distinct subgroups of human IgG designated IgG1, IgG2, IgG3 and IgG4. The four subclasses show more than 95% homology in the amino acid sequences of the constant domains of the heavy chains, but differ with respect to structure and flexibility of the hinge region, especially in the number of inter-heavy chain disulfide bonds in this domain. The structural differences between the IgG subclasses are also reflected in their susceptibility to proteolytic enzymes, particularly papain, plasmin, trypsin and pepsin.

In certain embodiments of any aspect of the invention, the Fc region is an Fc region of IgG4. Only one isoform of human IgG4 is known. In contrast to human IgG1, IgG2 and IgG3, human IgG4 does not activate complement. Furthermore, IgG4 is less susceptible to proteolytic enzymes compared to IgG2 and IgG3. Contrary to these expectations, it has surprisingly been found that, in practice, IgG1 full length antibody construct bearing mutations I253N and H435Q features lower affinities to FcRn as exemplified by the higher dissociation constants lower plasma to brain ratio determined compared to the corresponding IgG4 full length antibody construct.

Similarly within the scope of the present invention is a use of treating or preventing a malignant neoplastic disease, particularly a solid tissue tumor, more particularly glioma, in a patient in need thereof, comprising administering to the patient a polypeptide comprising a modified Fc region according to one of the aspects of the invention described above or a nucleic acid encoding the polypeptide or a viral vector comprising the nucleic acid encoding the polypeptide.

Similarly, a dosage form for the prevention or treatment of a malignant neoplastic disease, particularly a solid tissue tumor, more particularly glioma, is provided, comprising a polypeptide comprising a modified Fc region according to one of the aspects of the invention described above or a nucleic acid encoding the polypeptide or a viral vector comprising the nucleic acid encoding the polypeptide.

Wherever alternatives for single separable features are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

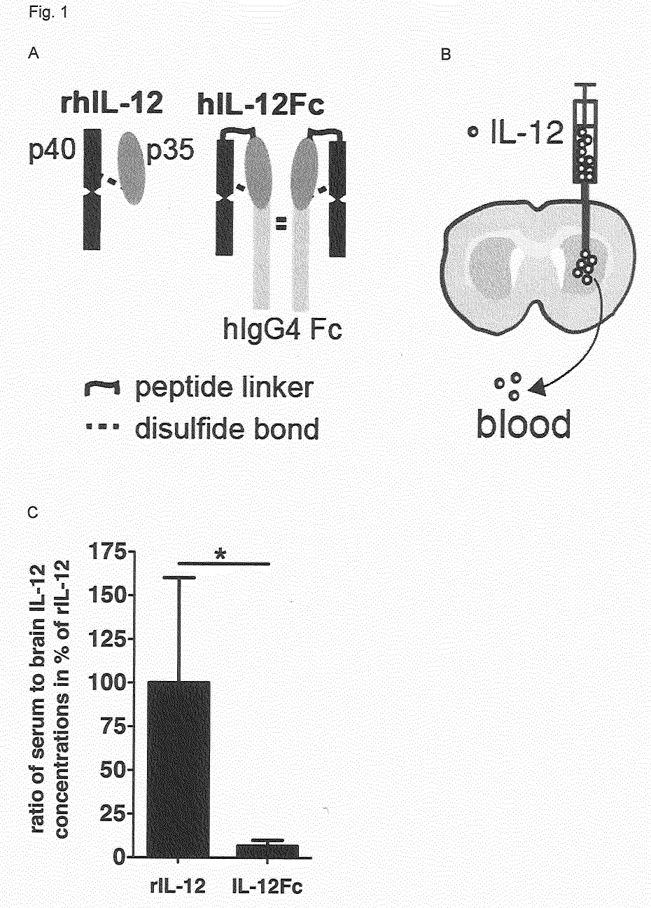

FIG. 1: Human IL-12Fc has better tissue retention than IL-12. A. Schematic structure of murine IL-12Fc. rhIL-12—recombinant human IL-12, hIL-12Fc—human IL-12Fc. IgG4 Fc—fragment crystallizable region of human IgG4. B. Schematic of the experiment. IL-12 of IL-12Fc was injected into the striatum of FcRn^{tg} mice. After 24 hours the remaining amount of injected protein was assessed in the brain and compared to the amount present in serum. C. Ratio of serum to brain IL-12 amount as assessed by ELISA. ELISA measured hIL-12 as a generic measure of IL-12Fc. Unpaired Student's t-test. **p<0.005. Mean±SD.

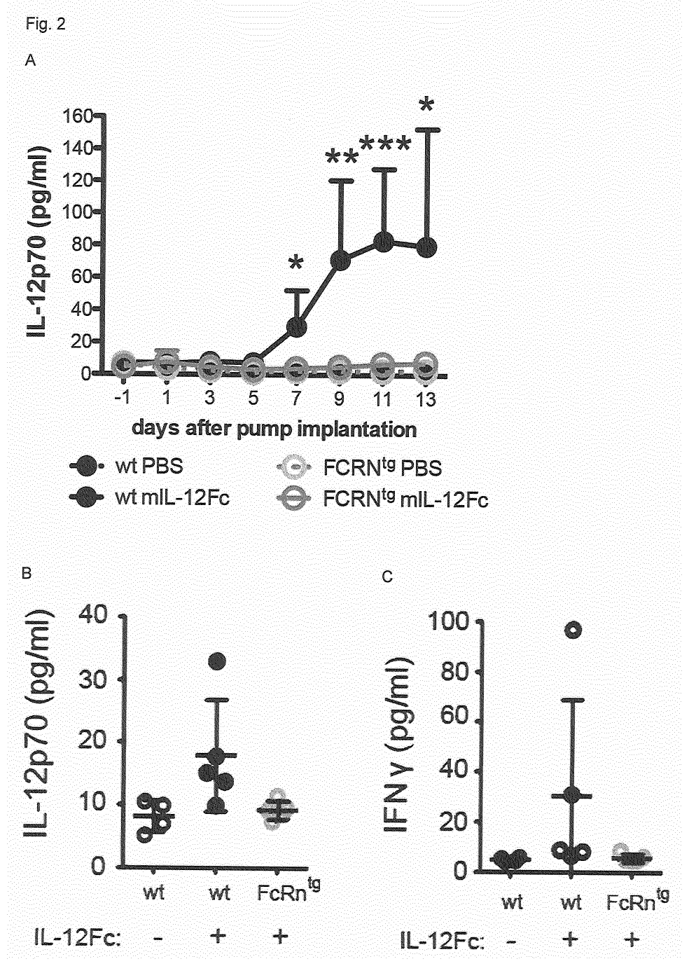

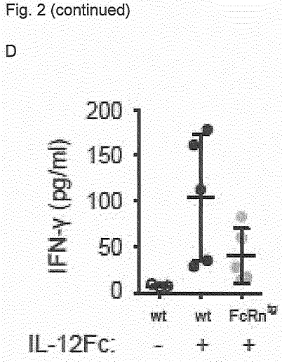

FIG. 2: IL-12Fc is being exported from the brain in an FcRn-mediated fashion. A. Brain tumor-bearing wt and FcRn^{tg} mice were implanted with osmotic pumps delivering 12.5 pg/kg/day of murine IL-12Fc directly into the tumor lesion. Murine IL-12 levels measured in serum using a bead-based array. Unpaired Student's t-test of groups wt mIL-12Fc vs FcRn^{tg} mIL-12Fc. Mean±SD. One way ANOVA with Tukey's multiple comparison test. B. Mice treated like in FIG. 2 A. Amount of IL-12 present in the circulation 24 hours after the start of the treatment as measured in serum using a bead-based array. Mean±SD. C. Mice treated like in FIG. 2 A. Levels of IFNγ in the circulation 24 hours after the start of the treatment as measured in serum using a bead-based array. Mean±SD. D. IFN-γ levels at day 7, experiment in A, Mean±SD.

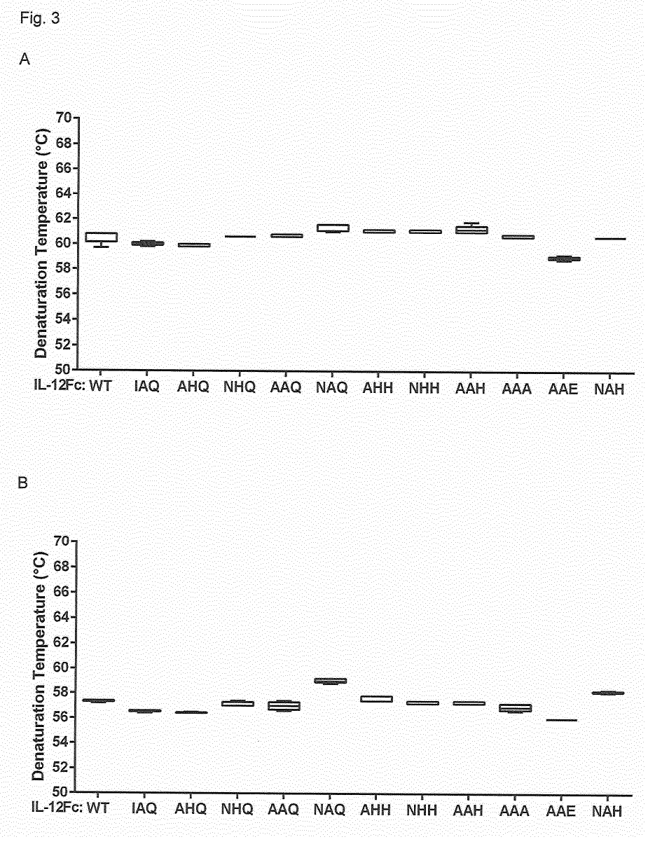

FIG. 3: Protein stability measured using thermal shift assay. Protein was incubated in PBS (A) or artificial cerebrospinal fluid (aCSF, B). Five measurements per IL-12Fc variant. Whiskers represent the minimum and maximum spread.

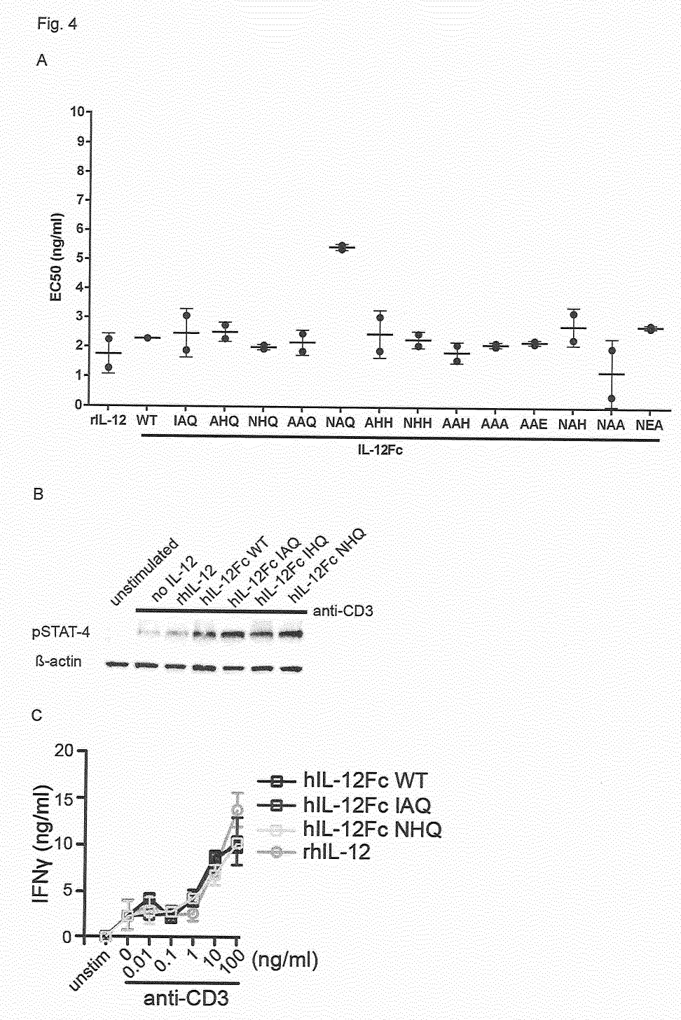

FIG. 4: Mutations in the Fc fragment of IL-12Fc do not affect the biological activity of IL-12. A. Bioactivity of IL-12 measured using HEK-BIue™ IL-12 assay. EC50—effective concentration leading to 50% of maximal signal from HEK-Blue™ IL-12 reporter cells stimulated with IL-12Fc in the range 0 to 50 ng/ml, two replicates per concentration. Measured by using the activity of the secreted alkaline phosphatase using a colorimetric method. Each point shows result from an independent experiment. Mean±SD. B. STAT-4 phosphorylation in peripheral blood mononuclear cells (PBMCs) stimulated for 1 h with 100 ng/ml anti-CD3 and 10 ng/ml of recombinant IL-12, IL-12Fc WT or three of the variants designed for reduced FcRn affinity. Mean±SD. C. IFNγ production by PBMCs stimulated for 24 h with 100 ng/ml anti-CD3 and indicated concentrations of recombinant IL-12, IL-12Fc WT or three of the variants designed for reduced FcRn Affinity.

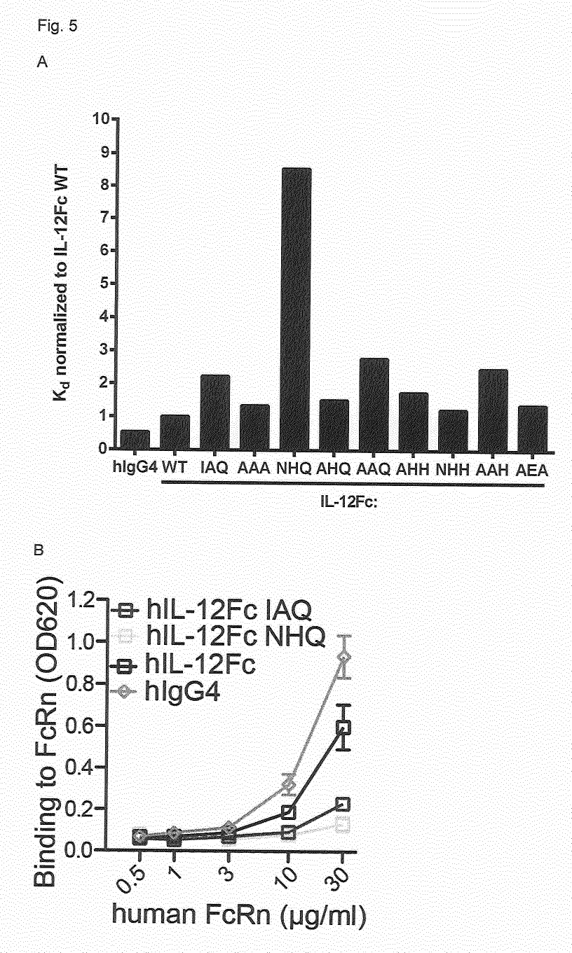

FIG. 5: Human IL-12Fc variants have reduced FcRn affinity. A. Surface plasmon resonance (SPR) measurement of FcRn affinity with human recombinant FcRn immobilized on the surface and IL-12Fc variants in the liquid phase. Affinity measured at pH=6.0. Data normalized to IL-12Fc WT. B. IL-12Fc variants binding to human FcRn. Measured by ELISA at pH=6.0. Mean±SD.

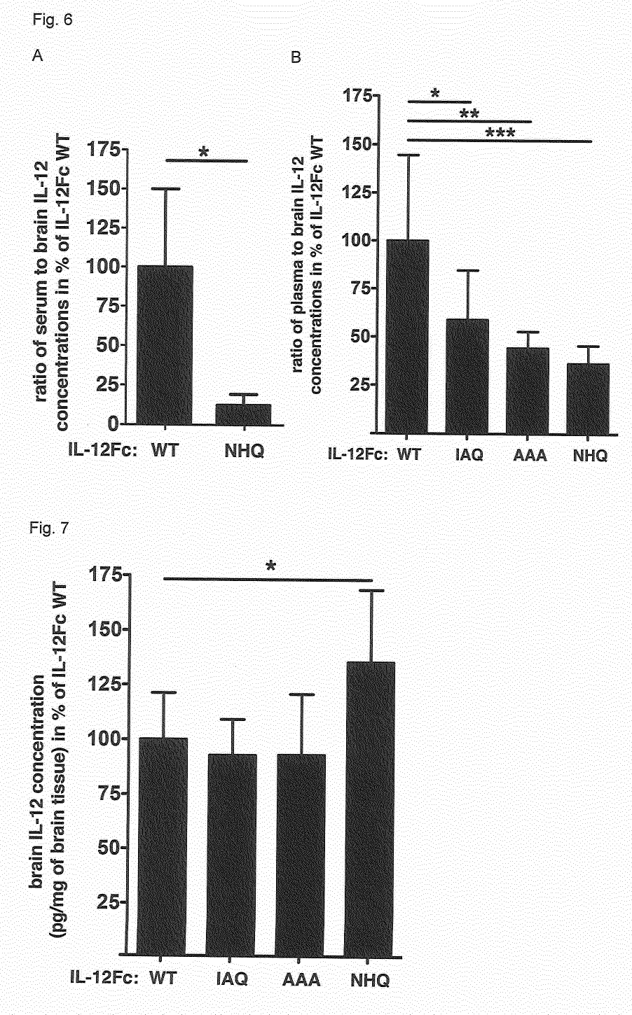

FIG. 6. Ratios of the concentrations of IL-12Fc in the blood and in the injected hemisphere. A. 1 pg of IL-12Fc WT or NHQ variant were injected into the striatum of FcRn^{tg} mice. After 24 hours the amounts of IL-12 were assessed in the injected brain hemisphere and in serum by ELISA, their ratios were calculated and normalized to those for IL-12Fc WT group. 4 mice per group. Unpaired Student's t-test. *p<0.05. Mean±SD. B. 1 pg of IL-12Fc WT, IAQ, AAA or NHQ were injected into the striatum of FcRn^{tg} mice using convection enhanced delivery (CED). After 24 hours the amounts of IL-12 were assessed in the injected brain hemisphere and in plasma by ELISA, their ratios were calculated and normalized to those for IL-12Fc WT group. 7-8 mice per group. One-way ANOVA with Tukey's multiple comparison test. Mean±SD.

FIG. 7: Brain retention after local treatment with IL-12Fc variants. FcRn^{tg} mice were injected with 1 μg of IL-12Fc WT, IAQ, AAA or NHQ into the striatum using convection enhanced delivery (CED). Amount of IL-12Fc remaining in the brain tissue was measured 6 hours after injection by ELISA and normalized to IL-12Fc WT. One-way ANOVA with Tukey's multiple comparison test. Outlier removal. Mean±SD.

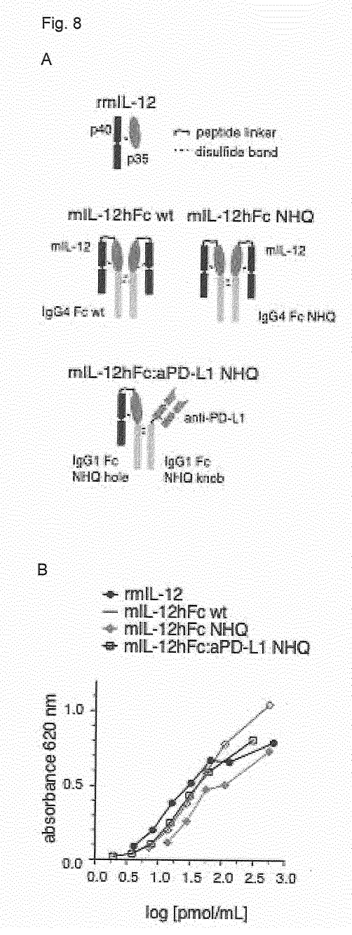

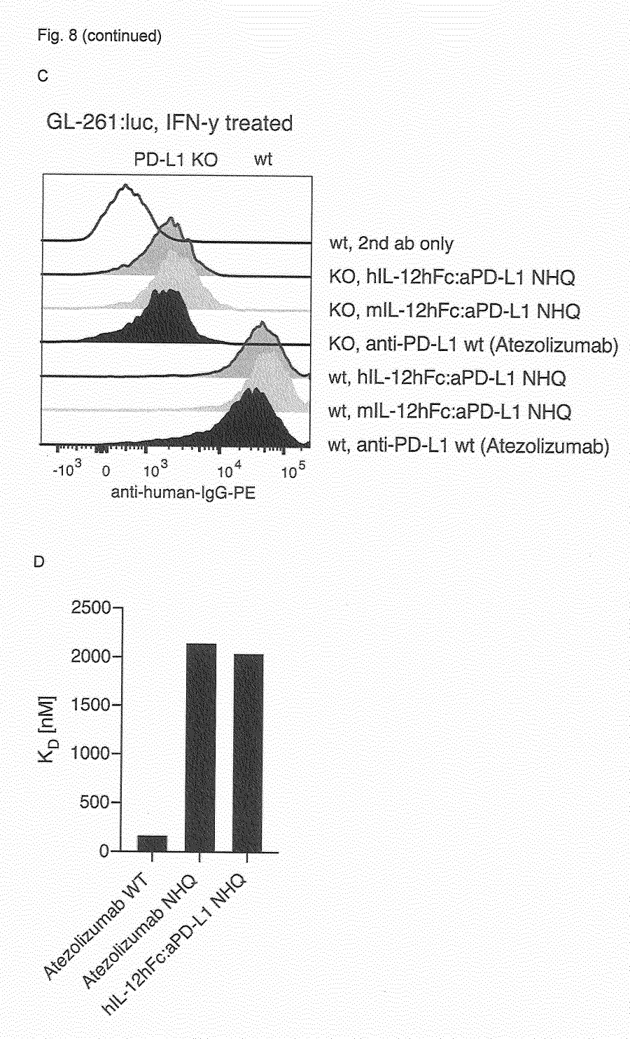

FIG. 8: A. Schematic structure of native and rmIL-12, mIL-12hIgG4 wt, mIL-12hIgG4 NHQ and mIL-12hIgG1:anti-hPD-L1 NHQ. B. Bioactivity of murine IL-12 constructs measured using HEK-Blue™ IL-12 assay. HEK-BIue™ reporter cells stimulated with IL-12 or IL-12Fc variants in the range of 0 to 50 ng/mL, using 5 to 8 dilution steps, two replicates per concentration. Measured by using the activity of the secreted alkaline phosphatase using a colorimetric method. X-axis values: concentration plotted as the corresponding amount of IL-12 molecules in pmol/ml. Representative of two individual experiments. C. Binding to PD-L1 on cells compared to full anti-PD-L1 (Atezolizumab) antibody. GL-261:luc or PD-L1 deficient GL-261:luc (PD-L1 KO) cells, stimulated with murine interferon-gamma (IFNγ) to stimulate PD-L1 expression, stained with anti-PD-L1 antibody or h/mIL-12hFc:aPD-L1 NHQ variants. Detection of cell-bound antibodies using anti-human-IgG-PE secondary antibody. D. Affinity to FcRn of NHQ mutated variants compared to WT as measured by surface plasmon resonance (SPR). Human recombinant FcRn immobilized on the surface and PD-L1 binders in liquid phase. Affinity measured at pH=6. Affinity constant K_{D} in nM.

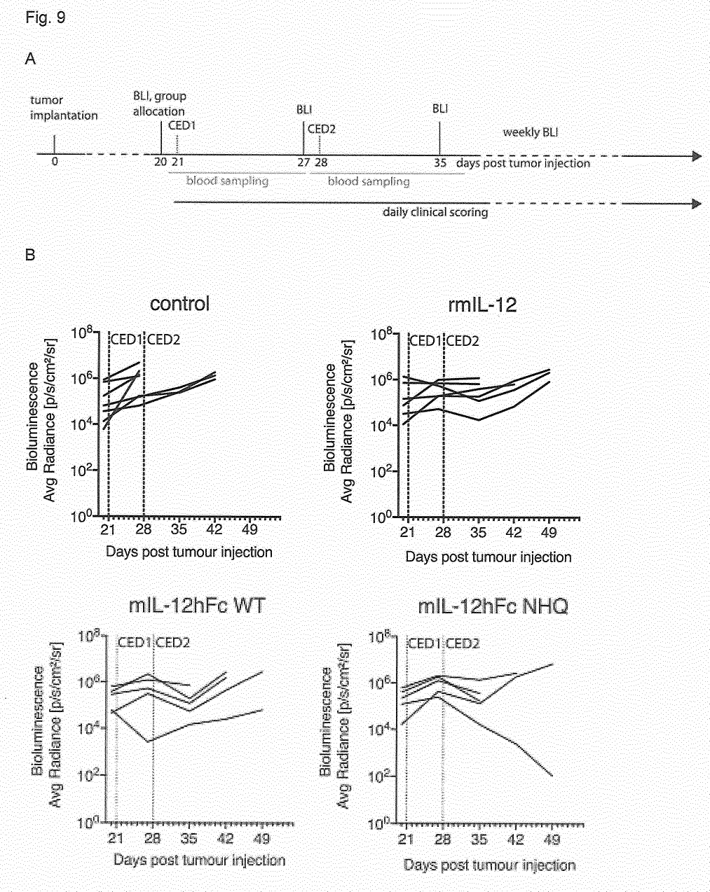

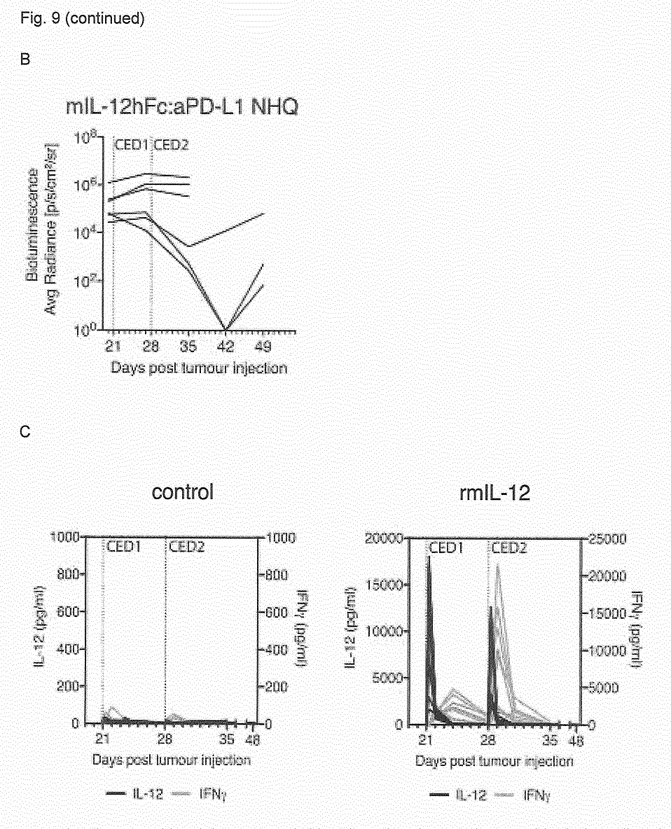

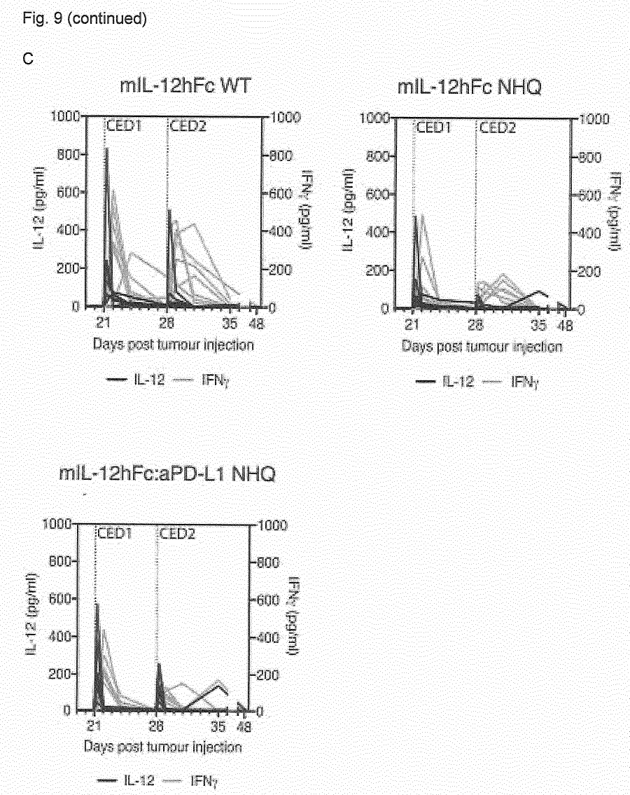

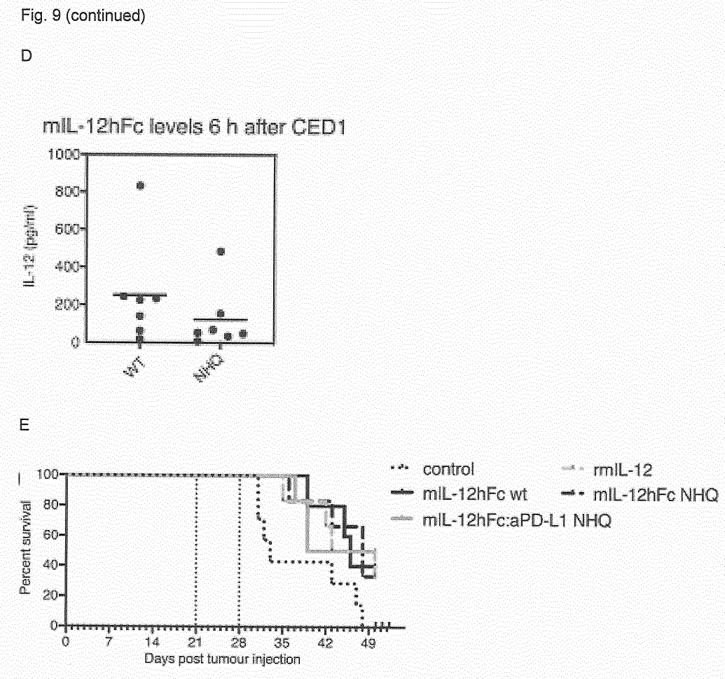

FIG. 9: Optimized IL-12 Fc fusions for local therapy of brain cancer lead to reduced systemic exposure without affecting the therapeutic effect.

A. Experimental timeline in days post tumor injection. GL-261:luc Brain tumor bearing animals were systematically allocated to treatment groups of comparable tumor load via bioluminescent imaging (BLI) on day 20 and treated via convection enhanced delivery (CED) with buffer only (control) or 1 pg of rmIL-12, mIL-12hFc:anti-PD-L1 bifunctional molecule, mIL-12hFc WT or mIL-12hFc NHQ on days 21 and d28 post tumor implantation. Blood sampling for plasma on time points: 0, 6 h, 24 h, 72 h, 7 days post CED injections as well as 14 days after the second CED injection.

B. Tumor progression upon treatment monitored by bioluminescence imaging. Plotted average radiance (p/s/cm2/sr) from region of interest (ROI) of individual animals, grouped by treatment cohort. Treatment via CED indicated by dotted vertical lines.

C. Plasma levels of IL-12 (black lines, left Y axis) and IFNγ (gray lines, right Y axis) in response to treatment. Measured on given time points by bead-based cytokine array. Treatment via CED indicated by dotted vertical lines.

D. FcRn affinity dependent difference of plasma IL-12 levels 6 h after CED on day 21. Mice injected with mIL-12hFc WT and mIL-12hFc NHQ. Data from experiment shown in A-C.

E. Kaplan-Meyer analysis of survival of treated mice from A-D. 6-7 mice per group.

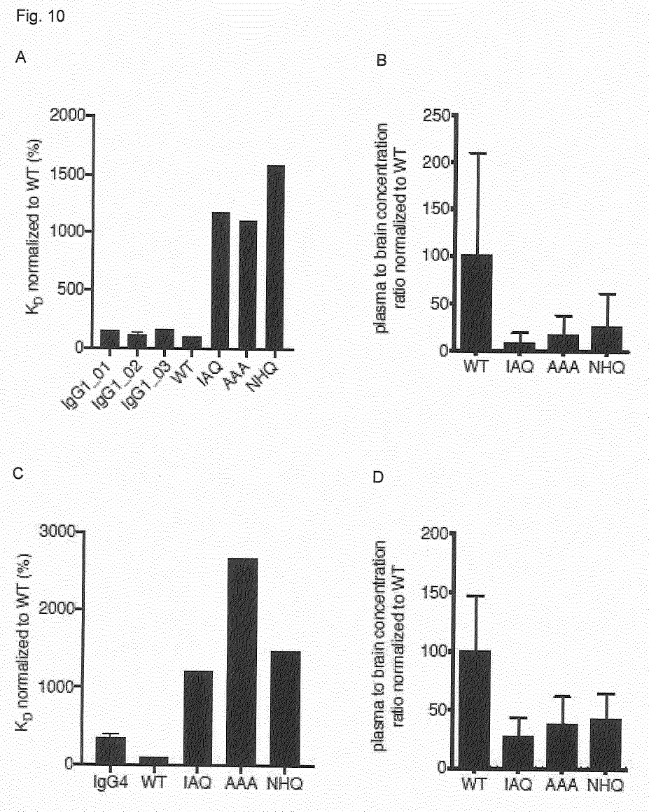

FIG. 10: Antibodies

A. Surface plasmon resonance (SPR) measurement of FcRn affinity with human recombinant FcRn immobilized on the surface and IgG1 variants in the liquid phase. Affinity measured at pH=6.0. Data normalized to an IgG1 antibody with a non-modified Fc (WT). Three additional IgG1 clinical grade antibodies with a non-modified Fc part were used as an additional reference (IgG1_01 ipilimumab, IgG1_02 atezolizumab, IgG1_03 rituximab). Mean±SD.

B. 1 μg of IgG1 WT, IAQ, AAA or NHQ variant were injected into the striatum of FcRn^{tg} mice using Convection Enhanced Delivery (CED). After 24 hours the amounts of human IgG were assessed in the injected brain hemisphere and in plasma by ELISA, their ratios were calculated and normalized to those for IL-12Fc WT group. 5 mice per group. Mean±SD.

C. Surface plasmon resonance (SPR) measurement of FcRn affinity with human recombinant FcRn immobilized on the surface and IgG4 variants in the liquid phase. Affinity measured at pH=6.0. Data normalized to an IgG4 antibody with a non-modified Fc (WT). A second IgG4 antibody (nivolumab) with a non-modified Fc part was used as an additional reference (IgG4). Mean±SD.

D. 1 pg of IgG4 WT, IAQ, AAA or NHQ variant were injected into the striatum of FcRn^{tg} mice using Convection Enhanced Delivery (CED). After 24 hours the amounts of human IgG were assessed in the injected brain hemisphere and in plasma by ELISA, their ratios were calculated and normalized to those for IL-12Fc WT group. 5 mice per group. Mean±SD.

### EXAMPLE 1: MATERIAL AND METHODS

Animals

C57BL/6J mice were obtained from Charles River. mFcRn^{-/-}hFcRn^{tg(32)} (FcRn^{tg}) mice were obtained from The Jackson Laboratory (stock number 014565). All animals were kept in house according to institutional guidelines under specific pathogen-free (SPH) conditions at a 12 h light/dark cycle with food and water provided ad libitum. All animal experiments were performed according to institutional guidelines and approved by the Swiss Cantonal Veterinary Office (license number 246/2015).

Tumor Cell Lines

GL-261 cells were provided by A. Fontana, Experimental Immunology, University of Zurich, Zurich, Switzerland and cultured in DMEM supplemented with 10% heat inactivated fetal calf serum and L-glutamine (all from Thermo Fisher Scientific). The murine GL-261 brain tumor cell line (syngenic to C57BL/6), was stably transfected with pGI3-ctrl and pGK-Puro (Promega) and selected with puromycin (Sigma-Aldrich) to generate luciferase-stable GL-261 cells. To generate GL-261:luc PD-L1 KO tumor cells, cells were transiently transfected with a Streptococcus pyogenes Cas9 P2A GFP—single guide RNA (sgRNA) expression vector (pX458; Addgene) modified to express the following sgRNA, 5′-GTATGGCAGCAACGTCACGA-3′. 3 days after transfection, GFP positive, PD-L1 KO cells were purified by flow cytometry by gating on PD-L1 negative cells after 48 h of IFN-γ stimulation (10 ng/ml). A single clone was further amplified and loss of PD-L1 expression was re-confirmed via flow cytometry before use in experiments.

Surgical Procedures

For glioma inoculation, 6-10 week old mice were anesthetized using a mixture of fentanyl (Helvepharm AG), midazolam (Roche Pharma AG) and medetomidin (Orion Pharma AG). GL261 cells were injected intracranially (i. c.) in the right hemisphere using a stereotactic robot (Neurostar). Briefly, a blunt-ended syringe (Hamilton; 75 N, 26 s/2″/2, 5 μl) was placed 1.5 mm lateral and 1 mm frontal of bregma. The needle was lowered into the burr hole to a depth of 4 mm below the dura surface and retracted 1 mm to form a small reservoir. Injection was performed in a volume of 2 μl at 1 μl/min. After leaving the needle in place for 2 min, it was retracted at 1 mm/min. The burr hole was closed with bone wax (Aesculap, Braun) and the scalp wound was sealed with tissue glue (Indermil, Henkel). Anesthesia was interrupted using a mixture of flumazenil (Labatec Pharma AG) and Buprenorphine (Indivior Schweiz AG), followed by injection of atipamezol 20 minutes later (Janssen). Carprofen (Pfizer AG) was used for perioperative analgesia.

After 7 to 14 days osmotic pumps (model 2004, 0.25 μl/h; Alzet) were filled with murine IL-12Fc (12.5 pg/kg/24 h) or PBS alone and primed at 37° C. in PBS. Mice were anaesthetized as above and the previous burr hole of the glioma injection was located, the bone wax and periosteal bone was removed, and the infusion cannula was lowered through the burr hole 3 mm into the putative center of the tumor. Serum samples were collected every two days by blood sampling from the tail vein, starting from day −1 of the pump implantation using Vacutainer tubes and following manufacturer's instructions (Becton, Dickinson and Company).

For the comparison of IL-12 and IL-12Fc WT serum to brain concentration ratio after bolus injection, mice were anesthetized and intracranially injected in the right hemisphere using a stereotactic robot (Neurostar) as described above for tumor cell injection. Mice received 100 ng of recombinant human IL-12 (Prospec) or equivalent amount of IL-12Fc (69 ng/mouse). Dosage was calculated based on the HEK-Blue IL-12 bioactivity assay. Animals were sacrificed 24 hours later by controlled CO₂ asphyxiation. Blood samples were collected by cardiac puncture and mice were perfused with 20 ml of ice cold PBS. Serum was isolated as described above and brain tissue was snap-frozen in liquid nitrogen.

For the comparison of IL-12 WT and IL-12Fc NHQ serum to brain concentration ratio after bolus injection, mice were anesthetized and intracranially injected in the right hemisphere using a stereotactic robot (Neurostar) as described above for tumor cell injection. Mice received 1 μg of human IL-12Fc WT or IL-12Fc NHQ. Animals were sacrificed 24 hours later by controlled CO₂ asphyxiation. Blood samples were collected by cardiac puncture and mice were perfused with 20 ml of ice cold PBS. Serum was isolated as described above and brain tissue was snap-frozen in liquid nitrogen.

For the convection enhanced delivery (CED) of protein into the brain, mice were anesthetized and intracranially injected in the right hemisphere using a stereotactic robot (Neurostar) and catheters made using a 27 G blunt-end needle with a 1 mm step at the tip made of fused silica with internal diameter of 0.1 mm and wall thickness of 0.0325 mm. Briefly, a burr hole was made at position 1 mm anteroposterior and 2 mm mediolateral of bregma. The catheter was lowered into the burr hole to a depth of 3.5 mm below the dura surface. Injection was performed in a volume of 5 μl at 0.2 μl/min, then 2 μl at 0.5 μl/min and 2 μl at 0.8 μl/min. After leaving the needle in place for 2 min, it was retracted at 1 mm/min. Mice received 1 μg of recombinant human IL-12Fc WT, IL-12Fc IAQ, IL-12Fc AAA, IL-12Fc NHQ or rmIL-12, mIL-12hFc WT, mIL-12hFc HNQ, mIL-12hFc:PD-L1 NHQ, Flu HA3.1 WT, Flu HA3.1 IAQ, Flu HA3.1 AAA, Flu HA3.1 NHQ or Atezolizumab WT, Atezolizumab IAQ, Atezolizumab AAA, or Atezolizumab NHQ. Animals were sacrificed 6 hours later by controlled CO₂ asphyxiation. Ipsilateral brain hemispheres were snap-frozen in liquid nitrogen.

In Vivo Bioluminescent Imaging

Tumor-bearing mice were injected with d-Luciferin (150 mg/kg body weight; XenoLight d-luciferin potassium salt; BioVision 7903-1G; 15 mg/mL in PBS). Animals were transferred to the dark chamber of a Xenogen IVIS Lumina III (PerkinElmer) imaging system and luminescence was recorded for 1 to 2 minutes, medium binning (4). Data were subsequently analysed using Living Image 4.7.1 software (PerkinElmer). A circular region of interest (ROI; 1.5 cm diam) was defined around the tumor site and photon flux of this region was read out and plotted.

BLI and Systematic Group Allocation

At d 20 after implantation of GL-261luc glioma cells, tumor-bearing animals were distributed into experimental groups of equal average BLI.

Blood Sampling

Blood samples were taken 10 min before CED or 6 h, 24 h, 72 h, 7 days after CED injections. 20 to 50 uL of blood were taken from the tail vein into a microtainer containing dried K2-EDTA (Becton, Dickinson and Company). After centrifugation for 5 min at 10′000 g, plasma was transferred to a fresh tube and frozen.

FcRn ELISA

IL-12Fc variants or a recombinant human IgG4 anti-GFP antibody (clone 515, AbD Serotec), which served as a control, were coated on a micro-well plate (Greiner Bio-One). Histidine-coupled FcRn (R&D Systems) was incubated at increasing concentration in ELISA diluent (Mabtech) at pH=6.0. FcRn was detected by a biotinylated anti-His-antibody (clone 13/45/31-2, Dianova), streptavidin-coupled horseradish peroxidase (Mabtech) and a colorimetric substrate (Chromogen-TMB, Thermo Fisher Scientific). Optical density at a wavelength of 450 nm was measured using a spectrophotometer (Molecular Devices).

Bead-Based Cytokine Array

Serum levels of mIL-12 and mIFNγ were measured using Legendplex Mouse Inflammation Panel (Biolegend) following manufacturer's instructions. Samples were acquired using LSRII Fortessa (Becton, Dickinson and Company). Data analysis was performed using FlowJo Version 10.6 (Tree Star).

HEK-Blue IL-12 Bioactivity Assay

HEK-Blue IL-12 cells (InvivoGen) were plated on a flat bottom 96 well plates (Corning) at a density of 50 000 cells/well in medium containing normocin (InvivoGen). Cells were incubated with increasing amounts of IL-12, IL-12Fc WT or variants designed for reduced FcRn affinity for 17 hours. Culture medium was collected and incubated for 2 hours in presence of Quanti-Blue detection reagent (InvivoGen). Absorbance was measured at 640 nm using a table top spectrophotometer (Molecular Devices).

Human IL-12 Detection in the Brain, Plasma and Serum after Injection into the Brain and Calculation of the Serum or Plasma to Brain Concentration Ratio

Samples were diluted in PBS containing 0.05% Tween-20 and 0.1% BSA and IL-12 levels were assessed by ELISA for hIL-12p70 (Mabtech). To calculate serum or plasma to brain concentration ratio, the concentration of IL-12 in serum or plasma was described in pg/ml, whereas concentration in brain was calculated by dividing the total amount of IL-12 extracted from the brain corrected for the efficacy of protein extraction by the weight of the hemisphere (pg/mg of brain tissue).

Human IgG Detection in the Brain and Plasma after Injection into the Brain and Calculation of the Plasma to Brain Concentration Ratio

Samples were diluted in PBS containing 0.05% Tween-20 and 0.1% BSA and IgG levels were assessed by ELISA. Briefly, plates were coated with polyclonal donkey anti-human IgG (Jackson ImmunoResearch), blocked with PBS containing 0.05% Tween-20 and 0.1% BSA. Analyte was detected with a polyclonal goat anti-human IgG (Sigma-Aldrich) and amplified with a polyclonal donkey anti-goat HRP-conjugated antibody (Promega). For calculation of the plasma to brain ratio, the concentration of both human IgG in plasma and brain was described in pg/ml.

Production of Human IgG1 Variants, IgG4 Variants hIL-12hFc:aPD-L1 NHQ and mIL-12hFc:aPD-L1 NHQ.

IgG4 variants were expressed in transiently transfected human embryonic kidney (HEK) cell cultures. IgG1 variants, hIL-12hFc:aPD-L1 NHQ and mIL-12hFc:aPD-L1 NHQ were produced by transiently transfected chinese hamster ovary (CHO) or cell cultures. Briefly, culture supernatants were collected and protein was purified by affinity chromatography (Protein G). Protein was further purified by ion exchange (IEC) and size Exclusion Chromatography (SEC). Protein was concentrated using spin columns (Sartorius, 30 kDa cutoff). Protein was stored in 20 mM histidine, 150 mM NaCl, pH=6.0 buffer. Quality was assessed by gel electrophoresis (SDS-PAGE) followed by Coomasie staining according to standard protocols. Nivolumab, atezolizumab, ipilimumab and rituximab are commercially available.

IFN-γ Production by Lymphocytes Stimulated with IL-12Fc

Human peripheral blood mononuclear cells (PBMCs) were stimulated for 24 h with increasing concentrations of IL-12, IL-12Fc or IL-12Fc variants with reduced FcRn affinity in presence of 100 ng/ml anti-CD3 antibody. IFN-γ levels in supernatant were measured by ELISA following manufacturer's instructions (Mabtech).

Brain Protein Isolation

After euthanasia and careful removal of the skullcap the brain was isolated. Cerebellum and olfactory bulbs were removed, the hemispheres are separated along the midline and the injected (ipsilateral) hemisphere was snap frozen in liquid nitrogen. Brain lysates were prepared by homogenization in ice cold lysis buffer (Cell signaling) containing Halt protease inhibitor cocktail (Thermo Fisher Scientific). 0.1 ml of lysis buffer was added per 10 mg of brain tissue. Brain tissue was minced using scissors, then passed through a 20 G needle and finally sonicated for 20 seconds. Samples were spun down for 10 minutes at 15000 g at 4° C. and supernatants were transferred to fresh tubes. Protein concentration was measured using Pierce BCA assay kit (Thermo Fisher Scientific) and this data was used to correct for the protein extraction efficiency within each experiment. Protein expression and purification

All human and murine IL-12Fc variants were expressed in HEK239T. Variants that retained protein G affinity, were purified from the culture supernatant by affinity chromatography using protein G sepharose (Biovision) and overnight dialysis with PBS. Variants that lost the protein G affinity were purified by precipitation with ammonium sulfate (VI) at 50% saturation, followed by dissolving the precipitate with PBS and purifying over ceramic hydroxyapatite (CHT) column (type II, 40 μm Bio-Rad). After protein G or CHT chromatography, samples were further purified by ion-exchange chromatography using diethylaminoethanol-linked sepharose as anionite (HiTrap DEAE Sepharose FF columns, GE Healthcare) on an ÄKTA Pure chromatography system (GE Healthcare). Finally, all the IL-12Fc variants were purified via size exclusion chromatography using a prepacked Superose 6 Column (GE Healthcare) on an ÄKTA chromatography system (GE Healthcare). Dimeric fraction was concentrated using Vivaspin 2 ml spin columns with 30 kDa cutoff (GE Healthcare). Protein purity was validated by SDS-PAGE electrophoresis followed by staining with Coomassie Brilliant Blue (VWR Life Science). Protein concentration was measured using Pierce BCA assay kit (Thermo Fisher Scientific) and by ELISA for IL-12p70 (Becton, Dickinson and Company).

Phosphorylation of STAT-4 by Lymphocytes Stimulated with IL-12Fc

Human peripheral blood mononuclear cells (PBMCs) were stimulated for 1 h with 10 ng/ml of IL-12, IL-12Fc or IL-12Fc variants with reduced FcRn affinity in presence of 100 ng/ml anti-CD3. Cells were then lysed using Pierce RIPA buffer (Thermo Fisher Scientific). Samples were analyzed by SDS-Page electrophoresis followed by transfer using Trans-Blot Turbo Blotting system (Bio-Rad Laboratories, Inc.) and staining with anti-STAT4 pY693 (clone 38/p-Stat4, Becton, Dickinson and Company). Band visualization was performed using ECL clarity substrate (Bio-Rad Laboratories, Inc.) and detection on BioRad MPCD imager (Bio-Rad Laboratories, Inc.).

Surface Plasmon Resonance

SPR was performed using the ProteOn XPR36 System (Bio-Rad Laboratories, Inc.), coating human recombinant biotinylated FcRn (Immunitrack) on a ProteOn NLC sensor chip to approximately 80 response units (RU). IL-12Fc variants were ran in 10 mM sodium citrate buffer pH=6.0 with decreasing concentration from 729 nM to 9 nM in three fold steps. The dissociation time was 600 s. Analysis was performed using the ProteOn Manager software (Bio-Rad Laboratories, Inc.) using data normalization to the injection time, interspot background removal and build-in artefact removal function. Kd were calculated using the equilibrium analysis model.

Thermal Shift Assay

Briefly, 0.2 mg/ml of protein samples were mixed with Sypro Orange Protein stain diluted to 1:1000 (Sigma-Aldrich) and ran on a CFX384 thermocycler (Biorad) with 0.2° C. temperature increase every 30 s from 20° C. to 95° C. with fluorescence as readout. Temperature of denaturation was determined as a first derivative of fluorescence over temperature. Experiment was performed in PBS and artificial cerebrospinal fluid (aCSF; 125 mM NaCl, 26 mM NaHCO₃, 1.25 mM NaH2PO₃, and 2.5 mM KCl) as solvents.

Statistical Analysis

Statistical analysis was performed using Graphpad Prism 5 software. Outliers were removed from the final analysis according to the Grubb's test (49). Two groups were compared using unpaired Student's t-test. More than two groups were compared using One-way ANOVA with Tukey's multiple comparison test.

Flow Cytometry PD-L1 Binding Assay

GL261:lucE9 or GL261:lucE9:PD-L1K0 cells were cultured over-night with addition of murine interferon-gamma in a final concentration of 20 ng/mL. The next day, cells were washed with DPBS. Trypsin-EDTA (Invitrogen 25300-054) was added to the flask and removed immediately again. Cells were left to detach from the flask for 2-5 min. They were washed with culture medium and centrifuged at 350×g 4° C. 5 min. Subsequently, cells were plated into a round-bottom 96-well plate at 100′000 cells per well and washed with DPBS twice.

Staining was performed in PBS, 25 μL per well containing Zombie Aqua fixable viability kit (BioLegend) diluted 1:200 and either human anti-PD-L1 (Atezolizumab) or m/hIL-12hFc:aPD-L1 NHQ at a final concentration of 0.1 mg/mL. Cells were stained for 20 min at 4° C. in the dark. Following a washing step with PBS, cells were incubated with secondary antibody anti-human IgG-Fc-PE (Biolegend, cat. nr. 409304, lot B260868) at 0.2 mg/mL or anti-mouse-PD-L1-BV421 (Biolegend, cat. nr. 124315; lot B228149) control antibody (data not shown), in PBS for 30 min at 4° C. in the dark. Cells were washed twice with PBS, filtered through a 40 μm mesh and acquired using LSRII Fortessa flow cytometer (BD). Data analysis was performed using FlowJo Version 10.6 (Tree Star).

Survival Analysis

Tumour-bearing animals were checked for neurological symptoms and weighed weekly until day 21 after tumour cell implantation. From day 21 onwards, monitoring frequency was increased to daily checks and weekly bioluminescence imaging (BLI). Animals were taken euthanized via controlled CO₂ asphyxation upon reaching predefined withdrawal criteria (weightloss over 20% of peak weight and/or moribund) according to cantonal veterinary authorities (ZH 194/19).

### EXAMPLE 2: INTRACRANIAL INJECTION OF HUMAN IL-12 HAS HIGHER SYSTEMIC LEAKAGE THAN HIL-12FC

IL-12Fc for the local treatment of brain tumors is very promising. However, for use in clinical trials a human version of IL-12Fc is required that needs to show similar properties. To obtain a human analogue to murine IL-12IgG3 the inventors fused single chain human IL-12 to the crystallisable fragment (Fc) of human immunoglobulin G4 (hIgG4) (FIG. 1A). Similar to mIgG3, hIgG4 does not support antibody dependent cell-mediated cytotoxicity (ADCC) and does not activate the complement system. To test for leakage and stability of human IL-12Fc (hIL-12Fc) vs recombinant human IL-12 (rhIL-12), the inventors injected a single bolus into the striatum of transgenic mice that express the human FcRn on a murine FcRn-deficient background (FcRntg) (Postow et al., 2015, N Engl J Med 372:2006-2017; Kamran et al., 2016, Expert Opin Biol Ther 16:1245-1264). After 24 hours, the inventors analysed the concentration of human IL-12 in the lysate of the ipsilateral hemisphere and in the serum to learn more about the stability and retention at the site of injection (residual concentration) and the rate of leakage into the bloodstream (FIG. 1B). For each mouse, the inventors calculated the ratios of concentrations in the serum vs the concentration at the injection site as an estimate for tissue retention. Comparing serum levels to local concentration at the injection site, hIL-12Fc showed superior tissue retention over rhIL-12, as the inventors observed considerably lower ratios (FIG. 1C). For local GB treatment, a human IL-12Fc fusion cytokine seems to be a superior compound compared to its natural counterpart due to its higher tissue retention, stability and solubility.

### EXAMPLE 3: FCRN BINDING LEADS TO SYSTEMIC ACCUMULATION OF IL-12FC

The neonatal Fc receptor (FcRn)-based endosomal recycling system in endothelial cells and red pulp macrophages prevents rapid degradation and clearance of IgG. After pinocytosis, facilitated by the acidic pH of endosomes, FcRn binds IgG and recycles it to the cell surface, where neutral pH induces its release. When injected locally, IL-12Fc can leak from the brain in an FcRn-mediated fashion due to its Fc tag. Leakage from the brain leads to IL-12Fc serum accumulation and may ultimately reach toxic levels. To test whether FcRn-based recycling indeed promotes accumulation of hIL-12Fc in the serum, the inventors utilized transgenic mice that express the human FcRn on a murine FcRn-deficient background (FcRntg). Since human FcRn has a weak affinity to murine IgG, but promotes normal albumin recycling, only murine IgG recycling is impaired in this mouse model. Murine IL-12Fc should therefore bind considerably less to FcRn in these FcRn humanized mice. Thus, the inventors compared serum mIL-12 levels of glioma-bearing wild type (wt) and FcRntg mice that were being treated with local murine IL-12Fc via osmotic minipumps. Indeed, after a week the inventors observed an increase in IL-12 levels in wt mice that was less pronounced in FcRntg mice (FIG. 2A) followed by an increase in IFN-γ levels (FIG. 2D). The inventors even observed elevated levels of IL-12 in the sera of some mice as early as day 1 after pump implantation (FIG. 2B). Consequently, this led to noticeable increase of IFN-γ serum levels in wt mice, which was not the case in the FcRntg cohort (FIG. 2C). Similar to murine IL-12Fc, human IL-12Fc will most likely also leak and accumulate, posing a threat for systemic side effects. Moreover, IFN-γ is one of the main mediators of the IL-12 related side effects (Leonard et al., 1997, Blood 90:2541-2548) and its persistent systemic presence can be toxic (Weiss et al., 2007, Expert Opin Biol Ther 7:1705-1721). Taken together, the inventors conclude that the leakage of even minute amounts of IL-12Fc from the treatment site is sufficient to trigger detectable serum IFN-γ levels.

### EXAMPLE 4: GENERATION OF HUMAN IL-12FC VARIANTS DESIGNED FOR IMPROVED TISSUE RETENTION

The observation that reduced FcRn binding potentially abrogates export from the brain and leads to dramatically reduced recycling upon leakage out of the brain can be exploited to increase the safety margin of hIL-12Fc. The inventors therefore set out to reduce the binding of the Fc portion of hIL-12Fc to human FcRn. By increasing the positive charge at the FcRn binding interface of the Fc part this interaction at acidic pH—and hence the recycling—can be abrogated, which was shown to decrease serum half-life of immunoglobulins. The inventors introduced a number of mutations into hIL-12Fc (Table 1), at the FcRn binding site of hIL-12Fc with the aim of decreasing its serum half-life in case of leakage.

The inventors have generated three IL-12Fc variants with mutations analogous to the previously published antibodies with reduced FcRn affinity called IAQ, AHH and AAA. Furthermore, the inventors substituted the isoleucine at position 253 not to alanine, which represents a simple shortening of the sidechain, but changed it to asparagine instead (I253N). Asparagine is a polar amino acid, whose sidechain has a similar length as isoleucine. The inventors have also modified histidine at position 310 to alanine and at position 435 to glutamine, alanine or glutamic acid.

All the variants were expressed in human embryonic kidney 293T cell (HEK293T) cultures. Expression levels for all the variants were similar.

### EXAMPLE 5: HUMAN IL-12FC VARIANTS HAVE SIMILAR PROTEIN STABILITY

First, the inventors have validated if the changes introduced to the Fc influence the overall protein stability. To this end the inventors have measured the denaturation temperature for each of the variant in a Thermal Shift Assay performed in PBS as well as in artificial cerebrospinal fluid (aCSF). The denaturation temperature for all the variants oscillated around 60° C. (FIG. 3 A). Measurements performed in aCSF confirmed that all the variants have similar stability, although the overall denaturation temperature was lower—approximately 57° C. (FIG. 3 B).

### EXAMPLE 6: HUMAN IL-12FC VARIANTS SUSTAIN THEIR BIOLOGICAL ACTIVITY

Even though the inventors aimed to reduce the binding of hIL-12Fc to FcRn the inventors could not exclude that those changes had influences on the IL-12 biological activity. This was tested first using a HEK cell line stably transfected with IL-12 signaling components and a downstream enzyme catalyzing a colorimetric reaction. Only the NAQ variant showed approximately 2× reduced activity compared to IL-12Fc, whereas all the others had an EC50 in the range of IL-12Fc WT (FIG. 4 A). Importantly, IL-12Fc had comparable activity in vitro as rIL-12.

To further validate activity of IL-12Fc variants, the inventors performed activation of peripheral blood mononuclear cells (PBMCs) with three different hIL-12Fc variants, namely IAQ, AHQ and NHQ, followed by analysis of STAT-4 phosphorylation (FIG. 4 B). More importantly, this STAT-4 phosphorylation translated into robust production of IFN-γ (FIG. 4 C) 24h later, indicating that all of the variants retained the activity of rhIL-12.

### EXAMPLE 7: HUMAN IL-12FC VARIANTS DIFFER IN BINDING TO PROTEIN G

Protein A and G affinity chromatography are among the standard methods used for purification of recombinant antibodies and Fc-fusion proteins. Modifying the interface between Fc and FcRn is known to abrogate Protein A binding, an observation that the inventors also confirmed with IL-12Fc variants. In order to confirm feasibility of production in a scaled-up process the inventors decided to validate the possibility of purifying IL-12Fc variants via Protein G affinity columns. The majority of the inventors' variants retained affinity to Protein G, but to the inventors' surprise, all the variants containing both I253N together with the H310A mutations were not suitable for protein G purification (Table 2). This effect was independent of additional mutations on position 435. For further studies the inventors have focused their attention on the variants with retained Protein G affinity.

### EXAMPLE 8: HUMAN IL-12FC VARIANTS HAVE REDUCED FCRN AFFINITY

In order to validate the affinity of the IL-12Fc variants to FcRn the inventors used surface plasmon resonance (SPR), a label-free method to characterize protein-protein interactions. The inventors immobilized human FcRn and measured the binding of IL-12Fc variants at lysosomal pH ranges (pH=6.0) in various concentrations (43). As shown on FIG. 5 A, the majority of the modified IL-12Fc variants have lowered affinity to human FcRn, with the NHQ variant showing the strongest reduction (approximately 8× lower). The inventors used a commercially available human monoclonal anti-GFP IgG4 antibody as a control.

Furthermore, the inventors corroborated these data with ELISA data for the NHQ construct using IL-12Fc WT, anti-GFP IgG4 and the published variant IAQ as references. Both IAQ and NHQ showed reduced binding, with the NHQ having the lowest affinity (FIG. 5 B). The inventors thus conclude that the NHQ combination of substitutions seemed to reduce the binding to FcRn most dramatically. This is in contrast to results from Kenanova and colleagues (Kenanova et al., 2005, Cancer Research 65:622-631) who suggested that the combined mutations H310A and H435Q are responsible for the strongest reduction of binding to FcRn at low pH.

### EXAMPLE 9: INTRODUCTION OF NHQ MUTATION REDUCES THE SYSTEMIC EXPOSURE TO LOCALLY DELIVERED HIL-12FC

The inventors hypothesized that reducing the FcRn affinity will increase the retention of hIL-12Fc in the CNS and at the same time prohibit its systemic accumulation. This was addressed in a similar fashion to the comparison of hIL-12Fc WT and recombinant human IL-12 (FIG. 1 B). FcRn^{tg} mice were injected with a single dose of 1 pg of IL-12Fc WT or the NHQ variant and IL-12 was measured by ELISA in the ipsilateral brain hemisphere and in serum. Mice injected with the NHQ variant showed lowered serum-to-brain ratios compared to mice injected with hIL-12Fc WT (FIG. 6 A). The inventors postulate that this can be an effect of both increased retention in the CNS as well as attenuated systemic accumulation due to the FcRn-mediated recycling.

Furthermore, using CED instead of bolus injection, the inventors have compared the concentrations of hIL-12Fc WT, IAQ, AAA and NHQ in plasma and the injected hemisphere 24 h after CED and observed that the NHQ variant exhibits the most significantly reduced plasma to brain ratio (FIG. 6 B) also in optimized delivery settings compared to bolus injection. Such increased CNS retention merged with lowered systemic exposure could potentially improve the safety profile of local IL-12Fc therapy.

### EXAMPLE 10: IL-12FC VARIANT NHQ HAS HIGHER BRAIN TISSUE RETENTION THAN OTHER LOW AFFINITY VARIANTS

Finally, the inventors measured the tissue retention after intracranial delivery of protein. To this end, the inventors injected 1 μg of unmodified IL-12Fc WT, the two previously published variants with reduced FcRn affinity, namely IAQ and AAA as well as NHQ, a variant with the lowest FcRn affinity according to the inventors' measurements (FIG. 5 A). In order to ensure maximal perfusion of the brain hemisphere, instead of a bolus injection of the protein solution, the inventors have used a CED protocol with a step catheter and a ramp-up injection regimen. To study the effect of different modifications at the FcRn binding interface in a most physiological setting, the inventors employed FcRn^{tg} mice. As discussed earlier, FcRn is important for both the export from CNS and accumulation of Fc-containing molecules in the serum. In an attempt to decouple the two effects and focus solely on preventing the transport from CNS, the inventors have measured the amount of protein left in the brain 6 hours after the CED. Mice were euthanized, perfused with PBS, total protein in the ipsilateral hemisphere was isolated and hIL-12 was measured by ELISA. As shown on FIG. 7, IL-12Fc NHQ has superior tissue retention compared to IL-12Fc WT. Importantly, it was also better than IAQ and AAA, the two other variants with reduced FcRn affinity. Surprisingly, IAQ and AAA were not significantly different than IL-12Fc WT.

### EXAMPLE 11: ANTI-TUMOR EFFECT IN VIVO

Human IL-12 is only poorly crossreactive with the murine IL-12 receptor. This implies, that for studying anti-tumor effect in vivo in a murine model, a surrogate molecule has to be used. In order to test the effects of the reduced affinity to FcRn, the inventors have fused single chain murine IL-12 to the same human IgG4 Fc as for hIL-12Fc (FIG. 8A).

IL-12 induces expression of IFNγ in target cells such as T cells and NK cells (Tugues et al., Cell death and differentiation (2015), 22:237-246)). IFNγ in turn can lead to upregulation of PD-L1 on myeloid cells and tumor cells in a process called adaptive resistance (O'Rourke et al., Sci. Transl. Med. (2017), (9), eaaa0984.). The inventors reasoned that PD-L1 would therefore serve as an induced anchor to further increase IL-12 tissue retention.

To assess the efficacy of IL-12Fc in combination with locally applied anti-PD-L1 antibody therapy, a bispecific Fc-fusion molecule was generated. It combines mIL-12hFc with an anti-PD-L1 half-antibody with a hIgG1 Fc containing NHQ mutation. The knobs-into-holes method was used for heterodimeric heavy chain assembly (Ridgway et al., Protein Eng (1996), 9:617-621). The anti-PD-L1 half-molecule is derived from atezolizumab, a clinically approved antibody and cross-reactive with murine and human PD-L1 (U.S. Pat. No. 8,217,149 B2) (FIG. 8A).

The inventors confirmed bioactivity of mIL-12hFc:aPD-L1 NHQ molecule in vitro: For IL-12 functionality, an IL-12-sensitive reporter cell line was used, IL-12 leads to secreted alkaline phosphatase, which in turn catalyzes a colorimetric reaction (FIG. 8B). Binding to cell bound PD-L1 was confirmed by flow-cytometry to detect binding of heterodimeric bifunctional constructs to PD-L1 on the surface of cells (FIG. 8C). The bifunctional heterodimeric constructs harbor the NHQ variant in their C_{H}2 and C_{H}3 domains and therefore FcRn binding is abrogated as confirmed by surface plasmon resonance and a comparably high K_{D} value compared to the unmodified anti-PD-L1 antibody (FIG. 8D).

Following the in vitro characterization, the inventors continued to measure its properties in vivo. Using a murine glioma model GL-261, anti-tumor effects and systemic distribution were monitored in vivo. Briefly, tumour-bearing mice received two intracranial injections via CED with rmIL-12, mIL-12hFc:aPD-L1 NHQ, mIL-12hFc WT or NHQ or vehicle control (injection buffer only) (FIG. 9 A). Changes in tumor size were monitored using bioluminescent imaging, clinical impact was monitored via clinical scoring (FIG. 9B). To assess leakage and export upon CED, systemic IL-12 and IFNγ levels were measured in the blood plasma at various points in time (FIG. 9C). While animals receiving rmIL-12 or mIL-12hFc wt exhibited a sharp increase of systemic IL-12 signal closely followed by IFNγ upon CED, animals receiving mIL-12hFc NHQ or mIL-12hFc:aPD-L1 NHQ showed strongly reduced systemic IL-12 signals which rapidly returned to baseline and markedly reduced IFNγ signals (FIG. 9C). The difference in tissue retention between mIL-12hFc wt and mIL-12hFc NHQ already 6 hours after CED1 leads lower systemic IL-12 signals (FIG. 9D). Regarding the clinical course of treated animals, all groups receiving IL-12 constructs showed a marked increase in survival (FIG. 9E) compared to the control group, even at exceptionally late timepoint of intervention when disease was far progressed, 3 weeks after tumor inoculation. Of note, treatment response in groups receiving NHQ constructs (mIL-12hFc NHQ or mIL-12hFc:aPD-L1 NHQ) showed a severely reduced systemic IL-12 and IFNg albeit responding at least equally well to treatment when compared to groups receiving mIL-12hFc wt or rmIL-12.

### EXAMPLE 12: AFFINITY MEASUREMENTS OF IL-12FC AND IGG VARIANTS TO HFCRN

To further evaluate the impact of low FcRn affinity on favorably influencing plasma to brain ratio upon local delivery to the CNS the IAQ, AAA and NHQ variants were compared to unmodified antibodies (FIG. 10). The inventors chose a human IgG1 directed against PD-L1 (FIG. 10A and FIG. 10B, atezolizumab,) and a human anti-Influenza A IgG4 antibody (FIG. 10C and FIG. 10D, Flu HA3.1, US2014/0370032A1).

The finding that hIL-12Fc is functional, has higher tissue retention than rhIL-12 and that abrogation of systemic recycling can increase the safety margin in case of leakage has potentially wide implications for the local administration of any Fc containing molecule. These modifications enable safe and efficacious local delivery of any antibody or Fc-fusion molecule for the local treatment of neurologic diseases.

Administration of therapeutics into the CNS via the systemic route (either per os or i.v.) is challenging mainly because of the BBB and—compared to the rest of the body—only a small selection of today's therapeutics actually reaches the brain. Unfortunately, antibodies and Fc containing biologics, particularly Fc-fusion proteins, do not readily cross the BBB and in addition are actively exported. Enabling transport of antibodies over the BBB into the brain parenchyma is being extensively studied, e.g. by exploiting receptor mediated transcytosis of transferrin. Cytokines have a short half-life in circulation and bear a high risk of adverse effects, which narrows their therapeutic opportunity window. Cytokines can be linked to antibodies homing to tumors, where they will accumulate, particularly NHS-IL-12. Even after subcutaneous dosing, these antibodies induce an IFNγ response as they travel to the tumor via the bloodstream. Initially, systemic delivery of IL-12 was assessed for treatment of non-brain cancers. However, these clinical trials had to be prematurely terminated, since—at effective doses—intravenous application led to serious adverse events, including deaths. One of the main reasons seems to have been the induction of IFNγ by IL-12.

The serum half-life and solubility of protein therapeutics can be improved by direct fusion of the therapeutic moiety with the crystallizable fragment (Fc) of antibodies. For direct local application in anatomically distinct locations this can lead also to less desirable effects. One of these can be FcRn-mediated export of Fc-containing molecules from immune privileged anatomical sites, particularly the brain and their serum accumulation analogous to IgG recycling.

The inventors have observed that local administration of an IL-12Fc fusion cytokine into the brain triggers FcRn dependent export of IL-12Fc through the BBB into the circulation. IL-12Fc accumulates in the blood and triggers potentially dangerous IFNγ production.

The inventors found that IL-12Fc with reduced FcRn affinity is functional and has higher tissue retention than recombinant IL-12, as well as unmodified IL-12Fc. When compared in a brain tissue retention experiment, the NHQ mutant was the only one with improved retention over IL-12Fc WT. Surprisingly, IAQ and AAA, two variants reported to have dramatically reduced FcRn binding were not different than unmodified IL-12Fc, suggesting that in order to obtain biological difference, the FcRn affinity must be reduced over a given threshold, that only the NHQ modification reaches. Alternatively, it cannot be ruled out that the NHQ mutations introduce other features that improve tissue retention in an FcRn-independent way.

This translates into an improved safety profile and broadens the therapeutic window for the IL-12Fc therapy of brain tumors. Moreover, the inventors' findings can be translated to any Fc containing therapeutics, primarily therapeutic antibodies where there is a strong rationale for local intracranial administration. Such an application route would be preferred due to weak efficacy when given systemically, potentially an effect of poor crossing through the BBB, or because the desired therapeutic effect should be contained locally. Local therapy with biologics optimized for such delivery should preclude the systemic toxicity and thus improve the safety profile of the drug.

TABLE 1
List of the mutations introduced to the Fc part of IL-12Fc. Amino acid
positions numbered according to EU numbering system (Edelman et al.
Proceedings of the National Academy of Sciences of the United States
of America (1969) 63(1):78-85).

| Name | SEQ ID NO. | Position 253 | Position 310 | Position 435 |
|---|---|---|---|---|
| Fc WT | SEQ ID NO. 001 | I | H | H |
| IAQ | SEQ ID NO. 002 | I | A | Q |
| AHQ | SEQ ID NO. 003 | A | H | Q |
| NHQ | SEQ ID NO. 004 | N | H | Q |
| AAQ | SEQ ID NO. 005 | A | A | Q |
| NAQ | SEQ ID NO. 006 | N | A | Q |
| AHH | SEQ ID NO. 007 | A | H | H |
| NHH | SEQ ID NO. 008 | N | H | H |
| AAH | SEQ ID NO. 009 | A | A | H |
| NAH | SEQ ID NO. 010 | N | A | H |
| NAA | SEQ ID NO. 011 | N | A | A |
| NAE | SEQ ID NO. 012 | N | A | E |
| AAA | SEQ ID NO. 013 | A | A | A |
| AAE | SEQ ID NO. 014 | A | A | E |

TABLE 2
List of IL-12Fc variants and their ability to bind to Protein G.

| | | Retained affinity to the Protein G |
|---|---|---|
| Name | SEQ ID NO. | chromatography bead: |
| Fc wt | SEQ ID NO. 001 | + |
| IAQ | SEQ ID NO. 002 | + |
| AHQ | SEQ ID NO. 003 | + |
| NHQ | SEQ ID NO. 004 | + |
| AAQ | SEQ ID NO. 005 | + |
| NAQ | SEQ ID NO. 006 | − |
| AHH | SEQ ID NO. 007 | + |
| NHH | SEQ ID NO. 008 | + |
| AAH | SEQ ID NO. 009 | + |
| NAH | SEQ ID NO. 010 | − |
| NAA | SEQ ID NO. 011 | − |
| NAE | SEQ ID NO. 012 | − |
| AAA | SEQ ID NO. 013 | + |
| AAE | SEQ ID NO. 014 | + |

TABLE 3
List of sequences of molecules, which combination makes a
bispecific antibody or antibody-like molecule binding to
human or mouse IL-12 receptor, in particular in an agonistic
manner, and human or mouse PD-L1.

| Name | SEQ ID NO. |
|---|---|
| mIL-12Fc-IgG4 NHQ Hole | SEQ ID NO. 015 |
| hIL-12Fc-IgG4 NHQ Hole | SEQ ID NO. 016 |
| mIL-12Fc-IgG1 short NHQ Hole | SEQ ID NO. 017 |
| hIL-12Fc-IgG1 short NHQ Hole | SEQ ID NO. 018 |
| mIL-12Fc-IgG1 long NHQ Hole | SEQ ID NO. 019 |
| hIL-12Fc-IgG1 long NHQ Hole | SEQ ID NO. 020 |
| anti-PD-L1 scFv-IgG4 NHQ Knob | SEQ ID NO. 021 |
| anti-PD-L1 scFv-IgG1 NHQ Knob | SEQ ID NO. 022 |
| anti-PD-L1 HC-IgG1 NHQ Knob | SEQ ID NO. 023 |
| anti-PD-L1 LC-IgG1 | SEQ ID NO. 024 |

A combined bispecific molecule may consist of molecules described as sequence SEQ ID NO 15 with SEQ ID NO 21, SEQ ID NO 16 with SEQ ID NO 21, SEQ ID NO 17 with SEQ ID NO 22, SEQ ID NO 18 with SEQ ID NO 22, SEQ ID NO 17 with SEQ ID NO 23 and SEQ ID NO 24, SEQ ID NO 18 with SEQ ID NO 23 and 24, SEQ ID NO 19 with SEQ ID NO 22, SEQ ID NO 20 with SEQ ID NO 22, SEQ ID NO 19 with SEQ ID NO 23 and SEQ ID NO 24, SEQ ID NO 20 with SEQ ID NO 23 and SEQ ID NO 24.

Items
1. A polypeptide comprising a crystallizable fragment (Fc) region of IgG, for use as a medicament (use in medicine), wherein
   said Fc region bears a modification resulting in reduced affinity to the neonatal Fc receptor (FcRn) and
   said polypeptide is delivered by local administration to the tissue affected by the disease.
2. The polypeptide for use as a medicament according to item 1, wherein the polypeptide is delivered
   by intracranial administration,
   by intrathecal administration, or
   by intraocular administration.
3. The polypeptide for use as a medicament according to any one of the above items, wherein the polypeptide is administered by intracranial administration and the serum or plasma to brain concentration ratio of said polypeptide is below a predetermined threshold selected from
   a. at most ⅔ of the serum or plasma to brain concentration ratio of the same polypeptide comprising a non-modified Fc region,
   b. at most ⅛ of the serum or plasma to brain concentration ratio of the same polypeptide neither comprising an Fc region nor peptide linkers,
measurable 24 h after intracranial bolus injection into the striatum of FcRn^{tg} mice.
4. The polypeptide for use as a medicament according to any one of the above items, wherein the polypeptide is administered by intracerebroventricular or intrathecal administration and the serum or plasma to CSF concentration ratio of said polypeptide is below a predetermined threshold selected from
   c. at most ⅔ of the serum or plasma to CSF concentration ratio of the same polypeptide comprising a non-modified Fc region,
   d. at most ⅛ of the serum or plasma to CSF concentration ratio of the same polypeptide neither comprising an Fc region nor peptide linkers,
measurable 24 h after intracerebroventricular or intrathecal injection of FcRn^{tg} mice.
5. The polypeptide for use as a medicament according to any one of the above items, wherein said reduced affinity of said polypeptide to FcRn is characterized by a dissociation constant (K_{D}) selected from
a. a K_{D} that is at least 2×, particularly at least 3×, more particularly at least 4×, even more particularly at least 5× increased compared to a K_{D} characterizing binding of FcRn to the same polypeptide comprising a non-modified Fc region, and
b. a K_{D} that is at least 1.5×, particularly at least 2× increased compared to a K_{D} characterizing binding of FcRn to the same polypeptide comprising a differently modified Fc region, namely one mutant selected from IAQ (bearing the mutations H310A and H435Q) and AAA (bearing the mutations I253A, H310A and H435A).
6. The polypeptide for use as a medicament according to any one of the above items, wherein intracranial delivery is effected by a method selected from
   a. single, intermittent or continous local infusion, including convection enhanced delivery (CED),
   b. intrathekal administration,
   c. in situ production of said polypeptide,
   d. release from implanted slow release formulations,
   e. molecular transport into the CNS,
   f. cellular transport into the CNS, or
   g. transport to the CNS after intranasal application.
7. The polypeptide for use as a medicament according to any one of the above items, for treatment or prevention of a disease that affects the central nervous system.
8. The polypeptide for use as a medicament according to item 7, wherein said disease affecting the central nervous system is a malignant disease, particularly a glioma, more particularly a high grade glioma (HGG).
9. The polypeptide for use as a medicament according to any one of the above items, wherein said Fc region is a human Fc region or a chimeric Fc region comprising a human amino acid sequence and bears a mutation at position 253, in particular I253A or I253N, more particularly I253N.
10. The polypeptide for use as a medicament according to item 9, wherein said Fc region does not bear a mutation at position 310.
11. The polypeptide for use as a medicament according to any one of the above items, wherein said Fc region comprises
   the mutations H310A and H435Q (IAQ);
   the mutations I253A and H435Q, and an H at position 310 (AHQ);
   the mutations I253N and H435Q, and an H at position 310 (NHQ);
   the mutations I253A, H310A and H435Q (AAQ);
   the mutations I253N, H310A and H435Q (NAQ);
   the mutation I253A and an H at position 310 and 435 (AHH);
   the mutation I253N and an H at position 310 and 435 (NHH);
   the mutations I253A and H310A, and an H at position 435 (AAH);
   the mutations I253N and H310A, and an H at position 435 (NAH);
   the mutations I253N, H310A and H435A (NAA);
   the mutations I253N, H310A and H435E (NAE);
   the mutations I253A, H310A and H435A (AAA); or the mutations I253A, H310A and H435E (AAE).
12. The polypeptide for use as a medicament according to any one of the above items, wherein said Fc region comprises
   the mutations I253N and H435Q, and an H at position 310 (NHQ);
   the mutations I253A, H310A and H435Q (AAQ);
   the mutations I253N, H310A and H435Q (NAQ);
   the mutations I253N, H310A and H435E (NAE); or
   the mutations I253A, H310A and H435E (AAE),
   particularly said Fc region comprises the mutations I253N and H435Q, and an H at position 310 (NHQ).
13. The polypeptide for use as a medicament according to any one of the above items, wherein said Fc region is or comprises a sequence characterized by SEQ ID NO 002 (IAQ), SEQ ID NO 003 (AHQ), SEQ ID NO 004 (NHQ), SEQ ID NO 005 (AAQ), SEQ ID NO 006 (NAQ), SEQ ID NO 007 (AHH), SEQ ID NO 008 (NHH), SEQ ID NO 009 (AAH), SEQ ID NO 010 (NAH), SEQ ID NO 011 (NAA), SEQ ID NO 012 (NAE), SEQ ID NO 013 (AAA) or SEQ ID NO 014 (AAE).
14. The polypeptide for use as a medicament according to any one of the above items, wherein said Fc region is or comprises a sequence characterized by SEQ ID NO 004 (NHQ), SEQ ID NO 005 (AAQ), SEQ ID NO 006 (NAQ), SEQ ID NO 012 (NAE) or SEQ ID NO 014 (AAE).
15. The polypeptide for use as a medicament according to any one of the above items, wherein said polypeptide is selected from
   a. a fusion protein comprising
      i. an effector polypeptide and
      ii. said Fc region; or
   b. an antibody or antibody-like molecule comprising said Fc region.
16. The polypeptide for use as a medicament according to any one of the above items, wherein said polypeptide is selected from
   a. a bispecific, trispecific or multispecific antibody or antibody-like molecule, particularly a bispecific antibody or antibody-like molecule specifically binding to
      i. CD3 and a tumor-associated antigen,
      ii. Histone and IL12 receptor in an agonistic manner;
      iii. PD-L1 and 4-1BB,
      iv. PD-L1 and CD28,
      v. PD-L1 and IL12 receptor in an agonistic manner, or
      vi. a tumor-associated antigen and IL12 receptor in an agonistic manner,
   b. an armed antibody or antibody-like molecule comprising an effector polypeptide, or
   c. a tumor conditional or tissue conditional antibody or antibody-like molecule comprising a shielding domain and a cleavable protease sensitive linker peptide.
17. The polypeptide for use as a medicament according to item 16, wherein said polypeptide is a bispecific, trispecific or multispecific antibody or antibody-like molecule, particularly a bispecific antibody or antibody-like molecule specifically binding to PD-L1 and comprising
   i. an effector polypeptide,
   ii. IL-12Fc,
   iii. a combination of a molecule being characterized by a sequence selected from SEQ ID NO. 015-016 and a molecule being characterized by a sequence of SEQ ID NO. 021,
   iv. a combination of a molecule being characterized by a sequence selected from SEQ ID NO. 017-020 and a molecule being characterized by a sequence of SEQ ID NO. 022; or
   v. a combination of a molecule being characterized by a sequence selected from SEQ ID NO. 017-020, a molecule being characterized by a sequence of SEQ ID NO. 023 and a molecule being characterized by a sequence of SEQ ID NO.024.
18. The polypeptide for use as a medicament according to item 16, wherein the polypeptide is administered by intracranial administration and the serum or plasma to brain concentration ratio of said polypeptide is below a predetermined threshold selected from
   a. at most ⅛ of the serum or plasma to brain concentration ratio of the same polypeptide comprising a non-modified Fc region,
   b. at most 1/20 of the serum or plasma to brain concentration ratio of the same polypeptide neither comprising an Fc region nor peptide linkers, measurable 24 h after intracranial bolus injection into the striatum of FcRn^{tg} mice.
19. The polypeptide for use as a medicament according to any one of items 15 to 18, wherein said effector polypeptide is selected from hIL-12, hIL-10, hIL-2, hIL-7, IFNα, IFNβ, IFNγ, hIL-15, TNFα, CTLA-4, TGFβ, TGFβII, GDNF, hIL-35, CD95, hIL-1RA, hIL-4, hIL-13, SIRPα, G-CSF, GM-CSF, OX40L, CD80, CD86, GITRL, 4-1BBL, EphrinA1, EphrinB2, EphrinB5, BDNF, C9orf72, NRTN, ARTN, PSPN, CNTF, TRAIL, IL-4, IL-3, IL-1, IL-5, IL-8, IL-18, IL-21, CCL5, CCL21, CCL10, CCL16, CX3CL1, and CXCL16, in particular said effector polypeptide is hIL-12.
20. The polypeptide for use as a medicament according to any one of items 15 to 18, wherein said antibody or antibody-like molecule is selected from an antibody or antibody-like molecule specifically binding to PD-L1, TNFα, Histone, IFNγ, CXCL10, CTLA4, PD-1, OX40 CD3, CD25, CD28, TREM2, IL-6, CX3CR1, CD25, Nogo-A, CD27, IL-12, IL-12Rb1, IL-23, CD47, TGFβ, EGFR, EGFRvIII, Her2, PDGFR, TGFR, FGFR, IL-4RA, TfR, LfR, IR, LDL-R, LRP-1, CD133, CD111, VEGFR, VEGF-A, Ang-2, IL-10, IL-10R, IL-13Rα2, α-synuclein, CSF1R, GITR, TIM-3, LAG-3, TIGIT, BTLA, VISTA, CD96, 4-1BB, CCL2, IL-1 or IL-1R, EphA2, EphA3, EphB2, EphB3, EphB4, LINGO-1, L1CAM, NCAM, SOD-1, SIGMAR-1, SIGMAR-2, TDP-43, Aβ, Tau, IFNα, IFNβ, TRPM4, ASIC1, VGCCs, CB₁, TTR, HTT, JCV, or C9orf72, in particular said antibody or antibody-like molecule is an antibody specifically binding to PD-L1, OX40, CD47 or Nogo-A.
21. An antibody or antibody-like molecule specifically binding to OX40 in an agonistic fashion comprising a crystallizable fragment (Fc) region of IgG, for use in prevention or treatment of a disease affecting the central nervous system, wherein
   said Fc region bears a modification resulting in reduced affinity to the neonatal Fc receptor (FcRn) and
   said antibody or antibody-like molecule is administered to the brain.
22. The antibody or antibody-like molecule for use in prevention or treatment of a disease affecting the central nervous system according to item 21, wherein the serum or plasma to brain concentration ratio of said antibody or antibody-like molecule is below a predetermined threshold is selected from
   a. at most ⅔ of the serum or plasma to brain concentration ratio of the same polypeptide comprising a non-modified Fc region,
   b. at most ⅛ of the serum or plasma to brain concentration ratio of the same polypeptide neither comprising an Fc region nor peptide linkers, measurable 24 h after intracranial bolus injection or CED into the striatum of FcRn^{tg} mice.
23. The antibody or antibody-like molecule for use in prevention or treatment of a disease affecting the central nervous system according to any one of items 21 to 22, wherein said reduced affinity of said antibody or antibody-like molecule to FcRn is characterized by a dissociation constant (K_{D}) selected from
   a. a K_{D} that is at least 2×, particularly at least 3×, more particularly at least 4×, even more particularly at least 5× increased compared to a K_{D} characterizing binding of FcRn to the same antibody or antibody-like molecule comprising a non-modified Fc region, and
   b. a K_{D} that is at least 1.5×, particularly at least 2× increased compared to a K_{D} characterizing binding of FcRn to the same antibody or antibody-like molecule comprising a differently modified Fc region, namely one mutant selected from IAQ (bearing the mutations H310A and H435Q) and AAA (bearing the mutations I253A, H310A and H435A)
24. The antibody or antibody-like molecule for use in treatment or prevention of a disease affecting the central nervous system according to any one of items 21 to 23, wherein said intracranial delivery is effected by a method selected from
   a. single, intermittent or continuous local infusion, including convection enhanced delivery (CED),
   b. in situ production of said polypeptide,
   c. intrathekal or intracerebroventricular administration,
   d. release from implanted slow release formulations,
   e. molecular transport into the cns,
   f. cellular transport into the CNS, or
   g. transport to the CNS after intranasal application.
25. The antibody or antibody-like molecule for use in treatment or prevention of a disease affecting the central nervous system according to any one of items 21 to 24, wherein said disease affecting the central nervous system is a malignant disease, particularly a glioma, more particularly a high grade glioma (HGG).
26. The antibody or antibody-like molecule for use in prevention or treatment of a disease affecting the central nervous system to any one of items 21 to 25, wherein said Fc region is a human Fc region or a chimeric Fc region comprising a human amino acid sequence and bears a mutation at position 253 [Kabat numbering system], in particular I253A or I253N, more particularly I253N.
27. The antibody or antibody-like molecule for use in prevention or treatment of a disease affecting the central nervous system according to item 26, wherein said Fc region does not bear a mutation at position 310.
28. The antibody or antibody-like molecule for use in prevention or treatment of a disease affecting the central nervous system according to any one of items 21 to 27, wherein said Fc region comprises
   the mutations H310A and H435Q (IAQ);
   the mutations I253A and H435Q, and an H at position 310 (AHQ);
   the mutations I253N and H435Q, and an H at position 310 (NHQ);
   the mutations I253A, H310A and H435Q (AAQ);
   the mutations I253N, H310A and H435Q (NAQ);
   the mutation I253A and an H at position 310 and 435 (AHH);
   the mutation I253N and an H at position 310 and 435 (NHH);
   the mutations I253A and H310A, and an H at position 435 (AAH);
   the mutations I253N and H310A, and an H at position 435 (NAH);
   the mutations I253N, H310A and H435A (NAA);
   the mutations I253N, H310A and H435E (NAE);
   the mutations I253A, H310A and H435A (AAA); or
   the mutations I253A, H310A and H435E (AAE).
29. The antibody or antibody-like molecule for use in prevention or treatment of a disease affecting the central nervous system according to any one of items 21 to 28, wherein said Fc region comprises
   the mutations I253N and H435Q, and an H at position 310 (NHQ);
   the mutations I253A, H310A and H435Q (AAQ);
   the mutations I253N, H310A and H435Q (NAQ);
   the mutations I253N, H310A and H435E (NAE); or
   the mutations I253A, H310A and H435E (AAE).
30. The antibody or antibody-like molecule for use in prevention or treatment of a disease affecting the central nervous system according to any one of items 21 to 29, wherein said Fc region is or comprises a sequence characterized by SEQ ID NO 002 (IAQ), SEQ ID NO 003 (AHQ), SEQ ID NO 004 (NHQ), SEQ ID NO 005 (AAQ), SEQ ID NO 006 (NAQ), SEQ ID NO 007 (AHH), SEQ ID NO 008 (NHH), SEQ ID NO 009 (AAH), SEQ ID NO 010 (NAH), SEQ ID NO 011 (NAA), SEQ ID NO 012 (NAE), SEQ ID NO 013 (AAA) or SEQ ID NO 014 (AAE).
31. The antibody or antibody-like molecule for use in prevention or treatment of a disease affecting the central nervous system according to any one of items 21 to 30, wherein said Fc region is or comprises a sequence characterized by SEQ ID NO 004 (NHQ), SEQ ID NO 005 (AAQ), SEQ ID NO 006 (NAQ), SEQ ID NO 012 (NAE) or SEQ ID NO 014 (AAE).

1-25. (canceled)

26. A polypeptide comprising:
a Fc region of IgG,
wherein said Fc region bears a modification resulting in reduced affinity to the neonatal Fc receptor (FcRn), and
said Fc region is or comprises a sequence characterized by SEQ ID NO. 002 (IAQ), SEQ ID NO. 003 (AHQ), SEQ ID NO. 004 (NHQ), SEQ ID NO. 005 (AAQ), SEQ ID NO. 006 (NAQ), SEQ ID NO. 007 (AHH), SEQ ID NO. 008 (NHH), SEQ ID NO. 009 (AAH), SEQ ID NO. 010 (NAH), SEQ ID NO. 011 (NAA), SEQ ID NO. 012 (NAE), SEQ ID NO. 013 (AAA) or SEQ ID NO. 014 (AAE).

27. The polypeptide according to claim 26, wherein said polypeptide is selected from:
a. a fusion protein comprising
i. an effector polypeptide, and
ii. said Fc region; or
b. an antibody or antibody-like molecule comprising said Fc region.

28. The polypeptide according to claim 27, wherein said effector polypeptide is selected from hIL-12, IL-10, IL-2, IL-7, IFNα, IFNβ, IFNγ, IL-15, TNFα, CTLA-4, TGFβ, TGFβRII, GDNF, hIL-35, CD95, IL-1RA, IL-4, IL-13, SIRPα, G-CSF, GM-CFS, OX40L, CD80, CD86, GITRL, 4-1BBL, EphrinA1, EphrinB2, and EphrinB5, BDNF, C9orf72, NRTN, ARTN, PSPN, CNTF, TRAIL, IFNα, IFNβ, IL-4, IL-3, IL-1 IL-5, IL-8, IL-18, IL-21, CCL5, CCL21, CCL10, CCL16, CX3CL1, and CXCL16.

29. The polypeptide according to claim 27, wherein said antibody or antibody-like molecule is selected from an antibody or antibody-like molecule specifically binding to a molecule selected from PD-L1, TNFα, Histone, IFNγ, CXCL10, CTLA4, PD-1, OX40, CD3, CD20, CD22, CD25, CD28, TREM2, IL-6, CX3CR1, Nogo-A, CD27, IL-12, IL-12Rb1, IL-23, CD47, TGFβ, EGFR, EGFRvIII, Her2, PDGFR, TGFR, FGFR, IL-4, IL-4RA, TfR, LfR, IR, LDL-R, LRP-1, CD133, CD111, VEGFR, VEGF-A, Ang-2, IL-10, IL-10R, IL-13Rα2, α-synuclein, CSF1R, GITR, TIM-3, LAG-3, TIGIT, BTLA, VISTA, CD96, 4-1BB, CCL2, IL-1 or IL-1R, EphA2, EphA3, EphB2, EphB3, and EphB4, LINGO-1, L1 CAM, NCAM, C0147, SOD-1, SIGMAR-1, SIGMAR-2, TDP-43, Aβ, Tau, IFNα, IFNβ, TRPM4, ASIC1, VGCCs, CB₁, TTR, HTT, JCV, and C9orf72.

30. The polypeptide according to claim 26, wherein said polypeptide is an antibody or antibody-like molecule comprising or linked to said Fc region.

31. The polypeptide according to claim 30, wherein said antibody or antibody-like molecule is a bispecific construct able to bind two antigens at the same time,

32. The polypeptide according to claim 31, wherein said bispecific antibody or antibody-like molecule binds to PD-L1 and IL-12 receptors in an agonistic manner.

33. A nucleic acid encoding the polypeptide according to claim 26.

34. A viral vector comprising the nucleic acid according to claim 33.

35. A method of treating a disease selected from brain cancer, stroke, dementia, Parkinson's disease, Alzheimer's disease, multiple sclerosis, epilepsy, and traumatic central nervous system injury, which comprises administering the polypeptide according to claim 26.

36. A method of treating a disease selected from uveal melanoma, uveitis, and wet macular degeneration, which comprises administering the polypeptide according to claim 26.

37. A method of treating a disease selected from rheumatoid arthritis, juvenile rheumatoid arthritis, gout, pseudogout, osteoarthritis, chronic hemophilic synovitis, psoriatic arthritis, and ankylosing spondylitis, which comprises administering the polypeptide according to claim 26.

38. A method of treating a disease selected from coronavirus disease 2019, diseased caused by severe acute respiratory syndrome coronavirus (SARS-CoV), severe acute respiratory syndrome, asthma, allergic asthma, severe uncontrolled asthma, fibrosis, cystic fibrosis, chronic obstructive pulmonary disease, influenza, lung oedema, sarcoidosis, lung cancer, tuberculosis, human orthopneumovirus, bubonic plague, pneumonic plague, anthrax, invasive fungal disease in lung, pulmonary fibrosis, respiratory syncytial virus, chronic rhinosinusitis with nasal polyps, interstitial lung disease, idiopathic pulmonary fibrosis, and pulmonary paracoccidioidomycosis, which comprises administering the polypeptide according to claim 26.

39. A method of preventing or treating a disease affecting a central nervous system (CNS), which comprises:
administering to a brain a fusion polypeptide comprising IL-12 and a crystallizable fragment (Fc) region of IgG,
wherein said Fc region bears a modification resulting in reduced affinity to the neonatal Fc receptor (FcRn).

40. The method according to claim 39, wherein a serum or plasma to a brain concentration ratio of said polypeptide is below a predetermined threshold selected from:
a. at most ⅔ of the serum or plasma to the brain concentration ratio of the same polypeptide comprising a non-modified Fc region, or
b. at most ⅛ of the serum or plasma to the brain concentration ratio of the same polypeptide neither comprising an Fc region nor peptide linkers, measurable 24 h after intracranial injection into the striatum of FcRn^{tg} mice.

41. The method according to claim 39, wherein said reduced affinity of said polypeptide to FcRn is characterized by a dissociation constant (K_{D}) selected from:
a. a K_{D} that is at least 2× increased compared to a K_{D} characterizing binding of FcRn to the same polypeptide comprising a non-modified Fc region, and
b. a K_{D} that is at least 1.5× increased compared to a K_{D} characterizing binding of FcRn to the same polypeptide comprising a differently modified Fc region, namely one mutant selected from IAQ and AAA.

42. The method according to claim 39, wherein said administration is effected by a method selected from:
a. single, intermittent or continuous local infusion, including convection enhanced delivery (CED),
b. intrathekal or intracerebroventricular administration,
c. in situ production of said polypeptide,
d. release from implanted slow release formulations,
e. molecular transport into the central nervous system,
f. cellular transport into the central nervous system, or
g. transport to the central nervous system after intranasal application.

43. The method according to claim 39, wherein said disease affecting the central nervous system is a malignant disease.

44. The method according to claim 43, wherein said malignant disease is a glioma or a high grade glioma (HGG).

45. The method according to claim 39, wherein said Fc region is a human Fc region or a chimeric Fc region comprising a human amino acid sequence and bears a mutation at position 253.

46. The method according to claim 45, wherein said mutation at position 253 is I253A or I253N.

47. The method according to claim 39, wherein said Fc region is or comprises a sequence characterized by SEQ ID NO. 002 (IAQ), SEQ ID NO. 003 (AHQ), SEQ ID NO. 004 (NHQ), SEQ ID NO. 005 (AAQ), SEQ ID NO. 006 (NAQ), SEQ ID NO. 007 (AHH), SEQ ID NO. 008 (NHH), SEQ ID NO. 009 (AAH), SEQ ID NO. 010 (NAH), SEQ ID NO. 011 (NAA), SEQ ID NO. 012 (NAE), SEQ ID NO. 013 (AAA) or SEQ ID NO. 014 (AAE).

48. A pharmaceutical composition suitable for use as a medicament, comprising:
a polypeptide comprising a crystallizable fragment (Fc) region of IgG,
wherein said Fc region bears a modification resulting in reduced affinity to the neonatal Fc receptor (FcRn), and
said Fc comprises mutations I253N and H435Q and an H at position 310.

49. The pharmaceutical composition according to claim 49, wherein said polypeptide further comprises IL-12.

50. The pharmaceutical composition according to claim 49, wherein said Fc region is or comprises a sequence SEQ ID NO. 004 (NHQ).

51. A method of preventing or treating a disease affecting the eye, which comprises:
administering to the eye by intraocular administration a polypeptide comprising a crystallisable fragment (Fc) region of IgG, wherein said Fc region bears a modification resulting in reduced affinity to the neonatal Fc receptor (FcRn), and said Fc comprises mutations I253N and H435Q and an H at position 310.

52. The method according to claim 51, wherein said polypeptide further comprises IL-12 or a polypeptide binding to any one of VEGFR, Ang2, TNFα, IL-17, PD-1 or PD-L1.

53. A method of preventing or treating a disease affecting a joint, which comprises:
administering to said joint by intraarticular administration a polypeptide comprising a crystallisable fragment (Fc) region of IgG, wherein said Fc region bears a modification resulting in reduced affinity to the neonatal Fc receptor (FcRn), and said Fc comprises mutations I253N and H435Q and an H at position 310.

54. The method according to claim 53, wherein said polypeptide further comprises IL-12 or a polypeptide binding to any one of TNFα, IL-1RA, IL-6R, IL-6, CD27, IL-22, IL-17 or CD27,

55. A method of preventing or treating a disease affecting the lungs, which comprises:
delivering to the lungs, via inhalation, a polypeptide comprising a crystallisable fragment (Fc) region of IgG, wherein said Fc region bears a modification resulting in reduced affinity to the neonatal Fc receptor (FcRn), and said Fc comprises mutations I253N and H435Q and an H at position 310.

56. The method according to claim 55, wherein said polypeptide further comprises IL-12 or IL-10 or a polypeptide binding to any one of IL-4RA, TNFα, IL-5, IL-6R, PD-1, PD-L1, CTLA-4, IL-8, IL-21R, CD25, CD20 or NF-kB.
